(19) United States
(12) Patent Application Publication
(10) Pub. No.: US 2004/0132107 A1
(43) Pub. Date: Jul. 8, 2004

(54) Functional assay for g-protein-coupled receptors based on insect cells

(76) Inventors: Thomas Anthony Grigliatti, Vancouver (CA); Peter John Kirk Knight, Suffolk (GB); Thomas Arthur Pfeifer, Vancouver (CA)

Correspondence Address: KNOBBE MARTENS OLSON & BEAR LLP, 2040 MAIN STREET, FOURTEENTH FLOOR, IRVINE, CA, 92614, US

(21) Appl. No.: 10/415,621

(22) PCT Filed: Oct. 26, 2001
(86) PCT No.: PCT/CA01/01516

(51) Int. Cl. G01N 33/53; G01N 33/567; C12N 5/06

(52) U.S. Cl. 435/7.2; 435/348

(57) ABSTRACT

The invention provides insect cell-based systems for assaying G-protein-coupled receptor interactions. The insect cells may express a heterologous mammalian G-protein-coupled receptor, a heterologous G alpha protein and one or more insect effector proteins that couple the heterologous G alpha protein to an endogenous insect signalling pathway. In such systems, binding of a ligand to the heterologous G-protein coupled receptor mediates a detectable change in the insect cell. The protein components of the system may be expressed from coding sequences that are stably maintained by transformed insect cells.

### FIELD OF THE INVENTION

The invention is in the field of molecular biology, particularly products and processes for measuring and testing molecular interactions of cell membrane bound receptors and ligands in animal (insect) cells, involving the use of recombinant nucleic acids.

### BACKGROUND OF THE INVENTION

Efficient cell-to-cell communication is crucial for the survival of multi-cellular organisms. This frequently entails the release of soluble, hydrophilic signaling molecules by one cell and the binding of these molecules by the cell surface receptors of another cell. Many of these receptors belong to the superfamily of G protein-coupled receptors (GPCRs), which are one of the largest protein families found in nature; there are estimated to be over 1,000 distinct GPCR genes present in the human genome. GPCRs are expressed in a range of human cell and tissue types, where they are understood to play a role in a broad array of physiological processes.

GPCRs are generally structurally related, with an extracellular N-terminus, 7 transmembrane domains, and an intracellular C-terminus. They are also thought to share a common mechanism of action. Binding of an extracellular ligand is thought to cause conformational changes in the GPCR that promote its interaction with heterotrimeric G-proteins on the inside face of the plasma membrane. There are multiple distinct classes of G-proteins, all described as consisting of alpha, beta and gamma subunits. Contact of a ligand bound receptor with a heterotrimeric G protein is understood to trigger exchange of GTP for GDP on the G alpha subunit, resulting in dissociation of the G-protein into alpha and beta-gamma subunits. Either or both of these components may then be able to interact with distinct effector enzymes or ion channels, whose actions trigger signal transduction cascades that may eventually modulate the expression of individual genes in the nucleus, thereby causing a physiological response by the cell to the original extracellular stimulus. Although the overall signaling paradigm is apparently the same for all GPCRs, the diversity of receptors, G proteins and effectors leads to a multitude of possible signaling processes and cellular responses¹. Signal transduction may be terminated in part by the intrinsic GTPase activity of G alpha, which hydrolyses bound GTP to GDP and so allows G alpha to reassociate with G beta gamma, returning the system to the resting state.

GPCRs are understood to be coupled to the cellular signaling machinery, such as calcium flux, through a variety of G alpha proteins, as shown in Table 1. Beta-gamma subunit pairs are also understood to enable GPCR coupling to the cellular signaling machinery, but their coupling specificity is less well understood.

TABLE 1
G alpha subunit families, and putative
cellular signaling mechanisms

| Gαs | | | |
|---|---|---|---|
| couple GPCRs | | Gαi | Gαq |
| to activation | | couple GPCRs | couple |
| of adenyly | Gα12 | to inactivation | GPCRs to |
| Icyclase, | coupling | of adenylyl | phospholipase C, |
| increase in | specificity | cyclase, | increase in |
| cAMP | not known | drop in cAMP | calcium |
| Gαolf | Gα12 | Gαo1 | Gα15 |
| Gαs | Gα13 | Gαo2 | Gα16 |
| | | Gαi1 | Gα11 |
| | | Gαi2 | Gαq |
| | | Gαi3 | Gα14 |
| | | Gαz | |

As a family, GPCRs are a major class of therapeutic targets; agents acting on GPCRs, either as agonists or antagonists, are widely used in drug therapy. GPCR-specific medicines have been developed for a range of cardiovascular, gastrointestinal, endocrine and metabolic diseases as well as autoimmune, CNS and inflammatory disorders². Given that it is generally believed that large numbers of GPCRs still remain to be identified in the human genome, it has been suggested that many of these will also be excellent therapeutic targets.

One of the earliest steps in many drug development programs targeting (newly discovered) human GPCRs is “primary screening”, which involves testing large collections of small molecule “ligands” (such as combinatorial chemistry libraries, chemical compound files and natural product libraries) for activity against the target receptor in an automated, high-throughput process. Primary or high throughput screening is generally dependent on having an efficient assay or test with which to measure the interaction between ligand and GPCR. Preferably, such an assay would be generic; that is, it should work with all (or nearly all) members of a particular class of receptor. In addition, it should preferably be functional, or mechanism-based, to facilitate identification of ligands that not only bind the GPCR, but also activate it. For this reason, traditional biochemical binding assays may be inadequate³. Attention has been focused on the development of cell-based, functional assays, in which the target receptor is expressed in an intact cell, coupled to an intracellular signal transduction system, which reports ligand-induced GPCR activation in an easily measurable form.

A cell-based, functional assay system for GPCRs using a target GPCR, G alpha₁₆ and apoaequorin has been constructed in a mammalian cell line²². Whilst mammalian cells may be useful for specific human GPCRs, they may be less useful as a generic host system because they tend to have a high background of endogenous GPCRs and G-proteins which can cause a high incidence of “false positive” results in screening²³. These may be expensive and time-consuming to analyze and eliminate. Mammalian cells may also be demanding and expensive to maintain, requiring elevated temperatures (37° C.) and CO₂, which may limit their useful life-span in a screening system.

Several assays for orphan GPCRs have been developed in other cellular backgrounds, most notably a yeast cell-based system²⁴. A yeast system is for example known that links GPCR activation to cell growth by substituting elements of an endogenous G-protein mediated pheromone response cascade with the heterologous GPCR. However, yeast cells may be unable to produce functionally active human GPCRs²⁵, making them a less attractive host for an assay system for assaying multiple human receptors whose functional expression is crucial but untestable. In addition, yeast cells typically possess a cell wall which is relatively impermeable, which may limit the accessibility of a ligand library to the expressed GPCR at the cell surface.

### SUMMARY OF THE INVENTION

In one aspect, the invention provides insect cell-based systems for assaying G-protein-coupled receptor interactions. The insect cells may express a heterologous G-protein-coupled receptor, such as a mammalian receptor, a heterologous G alpha protein, such as G alpha 15 or 16, and one or more insect effector proteins that couple the heterologous G alpha protein to an endogenous insect signalling pathway. In such systems, binding of a ligand to the heterologous G-protein coupled receptor mediates a detectable change in the insect cell. The protein components of the system may be expressed from coding sequences that are stably maintained by transformed insect cells.

In various aspects of the invention, there may be particular benefits associated with using insect cells, which fall evolutionarily somewhere between mammalian and yeast systems, combining many of the best features of both as a host system for GPCR functional assays. Like mammalian cells, the sophisticated expression machinery of insect cells has a high probability of producing functional, mature GPCRs. Unlike yeast cells, insect cells have no cell wall to limit access of ligand to the receptor. Like yeast cells, insect cells have unexpectedly been found to have a low background of endogenous GPCRs, and may therefore afford a relatively low likelihood of generating false positives during the screening process. The insect cell may for example be a lepidopteran cell, a dipteran cell, an Sf9 cell or a Hi5 cell.

In alternative embodiments, where the recombinant coding sequences utilized in the invention are stably retained in the insect cell lines, this may occur, for example, through integration into a chromosome, or by virtue of the stable replication and inheritance of a vector carrying the recombinant sequences.

The detectable change in the insect cells may for example be a calcium flux. The signalling pathway may comprise an endogenous insect phospholipase, such as phospholipase-C beta.¹⁷. A heterologous calcium binding protein, such as aequorin, may be expressed in the insect cell to provide the detectable signal, such as a bioluminescent signal, that is indicative of the calcium flux in the insect cell. The calcium binding protein may include a mitochondrial peptide targeting signal. A heterologous calcium ion channel may be expressed in the insect cell, such as a TRP ion channel transcribed from a Drosophila ion channel trp coding sequence.

In alternative embodiments, the insect cell may express a plurality of different heterologous G-protein-coupled receptors, each of which is coupled to the endogenous insect signalling pathway. The heterologous G-protein-coupled receptor may be a human G-protein-coupled receptor. The heterologous G alpha protein may be a G alpha 15 or G alpha 16 protein, in which case the heterologous G-protein-coupled receptor may even be one which couples in a natural state to a G protein other than G alpha 15 or G alpha 16. The heterologous G alpha protein may for example be selected from the group consisting of Gαolf, Gα12, Gαo1, Gα15, Gαs, Gα13, Gαo2, Gα16, Gαi1, Gα11, Gαi2, Gαq, Gαi3, Gα14, Gαz. The endogenous effector proteins comprise an endogenous insect G beta protein and an endogenous insect G gamma protein.

Methods of the invention for assaying G-protein-coupled receptor interactions, may include the step of exposing the insect cell to the ligand; and, detecting the detectable change in the insect cell. For example, a heterologous putative receptor ligand may be expressed in the insect cell from a putative receptor ligand coding sequence.

### BRIEF DESCRIPTION OF THE DRAWINGS

FIG. 1 is a schematic and conceptual outline of an insect cell-based assay for GPCRs. The illustrated interactions of the components of the system is for conceptual purposes only, and does not necessarily represent the actual interaction of any of the claimed embodiments of the invention (which is not limited to any particular mechanism of action). In the illustrated embodiment, the gene/protein components of the system include the human target GPCR, a human G-protein subunit (G alpha 16) and the Ca²⁺-sensitive bioluminescent protein aequorin. Agonist-induced activation of the GPCR results in activation of the Gq-type G protein formed between human G alpha 16 and endogenous insect beta gamma subunits. G alpha 16 is phosphorylated, dissociates from the insect beta gamma subunits and activates an endogenous effector enzyme, phospholipase-Cβ (PLCβ). Activated PLCβ cleaves membrane inositol phospholipids to release diacylglycerol (DAG) and inositol triphosphate (IP3), which in turn mediates the release of the second messenger Ca²⁺ from intracellular stores, principally the endoplasmic reticulum. Intracellular Ca²⁺ flux can be detected by co-expressing the Ca²⁺-sensitive photoprotein aequorin, which forms a bioluminescent complex when linked to the chromophore co-factor coelenterazine. In the presence of Ca²⁺, oxidation of bound coelenterazine leads to the production of light with a peak wavelength of 470 nm, which is detectable with a luminometer. After quantitation of agonist-specific Ca²⁺ flux, a detergent, such as Triton X100, may be used to solubilise the cell membrane, allowing an influx of Ca²⁺ and triggering complete exposure of the remaining (apo)aequorin. The results of the assay may be expressed as the ratio of agonist-induced luminescence to total (agonist-plus detergent-induced luminescence), called the fractional luminescence. Human genes/proteins are coloured black, other heterologous genes/proteins are coloured grey, and endogenous insect proteins are clear.

FIG. 2 shows a schematic for experimental determination of fractional luminescense(A/(A+L)); agonist response raw data, and method of reducing raw data to fractional luminescenseratio.

FIG. 3 shows a schematic for experimental determination of fractional luminescense (A/(A+L)); antagonist response raw data, and method of reducing raw data to fractional luminescense ratio.

FIG. 4 shows the effect of various antagonists on hDD1 assay response. To obtain the data, hDD1 insect-based assay cell lines were pre-incubated with various antagonists (0.1 microM cF, 0.1 microM SCH and 1 microM Hp) then challenged with 10 microM dopamine. Fractional luminescense responses for each antagonist (black bars above) were plotted as a percentage of the maximal response in the absence of antagonists (clear bar above). The data were pooled from two independent experiments, in each of which n=2. Dop=dopamine, the in vivo agonist for the hDD1 GPCR. Anatgonists are as follows; cF-cisFlupenthixol, SCH SCH23390, Hp=Haloperidol.

FIG. 5 shows concentration-activity curves for the agonists dopamine and SKF38393 versus the human Dopamine D1A GPCR in DADR insect assay cell lines. Each point represents the mean value of triplicate experiments, +/−the standard error of the mean.

FIG. 6 shows inhibition of the activity of 100 microM dopamine against the human Dopamine D1A GPCR in DADR insect assay cell lines by increasing concentrations of the antagonists cis Flupenthixol and Haloperidol. Each point represents the mean value of triplicate experiments, +/−the standard error of the mean.

FIG. 7 shows the effect of substituting G_{—15} for G_{—16} in dopamine D1A (DADR) insect assay cell lines. Activity of 100_M dopamine or growth media (ESF) against DADR insect assay cell lines transformed with Dopamine D1A, G_{—16}, and Aequorin (D1/G16/Aq), or Dopamine D1A, Gα₁₅, and Aequorin (D1/G15/Aq),), or Dopamine D1A and Aequorin (D1/Aq), or Ga₁₆ and Aequorin (G16/Aq). Each bar represents the mean value of six experiments, +/−the standard error of the mean.

FIG. 8 shows concentration-activity curves for the agonist dopamine versus the human Dopamine D1A GPCR in DADR insect assay cell lines constructed using either Sf9 or Hi5 insect cell lines. Each point represents the mean value of triplicate experiments, +/−the standard error of the mean.

FIG. 9 shows concentration-activity curves for the agonist U44069 versus the human Thromboxane A2 GPCR in TA2R insect assay cell lines. Each point represents the mean value of triplicate experiments, +/−the standard error of the mean.

FIG. 10 shows inhibition of the activity of 10 microM U44069 versus the human Thromboxane A2 GPCR in TA2R insect assay cell lines by increasing concentrations of the antagonist SQ29,548. Each point represents the mean value of triplicate experiments, +/−the standard error of the mean.

FIG. 11 shows concentration-activity curves for the endogenous agonist histamine versus the Histamine H1 GPCR in HH1R insect assay cell lines. Each point represents the mean value of triplicate experiments, +/−the standard error of the mean.

FIG. 12 shows inhibition of the activity of 100 microM histamine versus the human Histamine H1 GPCR in HH1R insect assay cell lines by increasing concentrations of any of the H1-spcific antagonists antagonists ketotifen fumarate, mepyramine maleate or triprolidone hydrochloride. Each point represents the mean value of triplicate experiments, +/−the standard error of the mean.

FIG. 13 shows transiently transformed insect assay cell lines expressing various combinations of HH1R (H1), Galpha₁₆ (G16) and aequorin (Aq) as indicated. “his”=100 microM histamine (agonist). “trip+his”=10 mM triprolidone hydrochloride (antagonist) followed by 100 microM histamine (agonist). “ESF”=growth media (negative control). Results are expressed as the mean value from triplicate experiments, +/−the standard error of the mean.

FIG. 14 shows concentration-activity curves for the endogenous agonist serotonin versus the 5-HT2A GPCR in 5H2A insect assay cell lines. Each point represents the mean value of triplicate experiments, +/−the standard error of the mean.

FIG. 15 demonstrates the ability to multiplex GPCRs in insect assay cell lines. The experiment compares insect assay cell lines containing human Dopamine D1A GPCR (D1 line), human 5-HT2A GPCR (5HT2A line) and both GPCRs co-transfected into the same assay cell line (dual line). Individual lines were incubated with the agonists dopamine (dop; 1 mM) or serotonin (5ht; 1 mM), or with the antagonists cis-Flupenthixol (cF; 1 microM), followed by dopamine (dop; 1 mM), or loxapine (lox; 10 microM), followed by serotonin (5ht; 1 mM). Results are expressed as the mean value of triplicate experiments, +/−the standard error of the mean.

FIG. 16 shows transiently transformed insect assay cell lines expressing various combinations of 5H1A(1a), Galpha₁₆ (G16) and aequorin (Aq) as indicated. “5ht”=100 microM serotonin (agonist). “dlp+his”=10 mM DL-propranalol (antagonist) followed by 100 microM serotonin (agonist). “ESF”=growth media (negative control). Results are expressed as the mean value of triplicate experiments, +/−the standard error of the mean.

FIG. 17 shows transiently transformed insect assay cell lines expressing various combinations of B2AR(b2AR), Galpha₁₆ (G16) and aequorin (Aq) as indicated. “na”=100 microM nor-epinephrine (agonist). “dlp+na”=10 mM DL-propranalol (antagonist) followed by 100 microM nor-epinephrine (agonist). “ESF”=growth media (negative control). Results are expressed as the mean value of triplicate experiments, +/−the standard error of the mean. This data illustrate

FIG. 18 shows data from transiently transformed insect assay cell lines expressing various combinations of ACM1 (M1), Galpha₁₆ (G16) and aequorin (Aq) as indicated. “ac”=100 microM acetylcholine (agonist). “bms+ac”=10 benztropine methanosulphate (antagonist) followed by 100 microM acetylcholine (agonist). UESF”=growth media (negative control). Results are expressed as the mean value of triplicate experiments, +/−the standard error of the mean.

### DETAILED DESCRIPTION OF THE INVENTION

In one aspect, the invention provides an insect cell-based, functional assay system that can be applied to the analysis of a wide variety of target GPCRs (including putative human GPCRs), regardless of their normal signal transduction mechanism. In some embodiments, the invention provides a test or screening method that may be used to identify agonist or antagonist molecules to a target GPCR from chemical files, combinatorial chemistry compound libraries, natural product libraries, cell, tissue or organ extracts, or other collections of compounds or molecules.

In one aspect, the assay of the invention involves co-expressing a human GPCR, a human G protein alpha-subunit, and a Ca²⁺-sensitive bioluminescent reporter protein in insect tissue culture cell lines. When the human GPCR is activated by an applied ligand, it couples via the human G protein alpha-subunit to an insect signal transduction system that generates a Ca²⁺ flux, which in turn triggers a detectable flash of light from the bioluminescent protein. In such embodiments, the host cells are insect tissue culture cell lines that can be permanently transformed with human genes.

In one aspect of the invention, it has unexpectedly been discovered that a variety of GPCRs which are understood to be coupled to cellular signaling mechanisms through various G alpha proteins, such as G alpha s, G alpha q or G alpha 1, may be coupled to the calcium flux response in insect cells through G alpha 15 and/or G alpha 16 proteins. This is due to the unexpected ability of G alpha 15 and/or G alpha 16 subunits to interact with endogenous insect G beta and G gamma subunits to form a functional heterotrimeric G protein. Accordingly, in one aspect the present invention provides an insect cell system for assaying heterologous GPCR interactions mediated by a heterologous G alpha 15/16 protein. Such a system of the invention may be amenable to use with a wide variety of GPCRs, including GPCRs that are normally coupled to other G alpha proteins. In some embodiments, the use of such a system will facilitate the analysis of a wide variety of GPCRs without the necessity of identifying and utilizing a specific G alpha protein with which the GPCR normally interacts. This aspect of the invention may be particularly advantageous in assaying the interactions of newly identified GPCRs, for which the particular G alpha protein affinity is uncharacterized. Examples of instances wherein a G alpha 15 or 16 protein has been shown to couple a GPCR to an endogenous insect signaling mechanism (calcium flux) are shown in Table 2.

TABLE 2
Gα15/16 coupled GPCRs in assays of the invention

| | Normally |
|---|---|
| GPCR | Coupled To |
| Dopamine D1A | G alpha s |
| Thromboxane A2 | G alpha q |
| 5-HT1A | G alpha i |

Extensive research with the insect Drosophila as a whole animal model system has indicated that this invertebrate shares many basic biological processes with humans⁴, including GPCR signal transduction mechanisms. Nevertheless, recent analysis of the complete Drosophila genome suggests, rather surprisingly, that they have far fewer GPCRs and G proteins (both in absolute number and as a proportion of their genome) than both humans and C. elegans⁵. It is reasonable to conclude that the relatively closely related lepidopteran invertebrates may also have comparatively few GPCRs and G proteins, and this is supported by a number of studies with Sf9 lepidopteran cell lines⁶. The paucity of GPCRs and G proteins could be taken to suggest that insect cells would be a poor system in which to assay GPCR interactions. However, in accordance with some embodiments of the invention, this feature of insect cells is utilized to provide a GPCR screening system with reduced probability of having false positive signals from endogenous GPCRs. At the same time, it has been discovered that the insect cell G protein signaling system is surprisingly promiscuous in its ability to produce a detectable response to the stimulation of heterologous GPCRs.

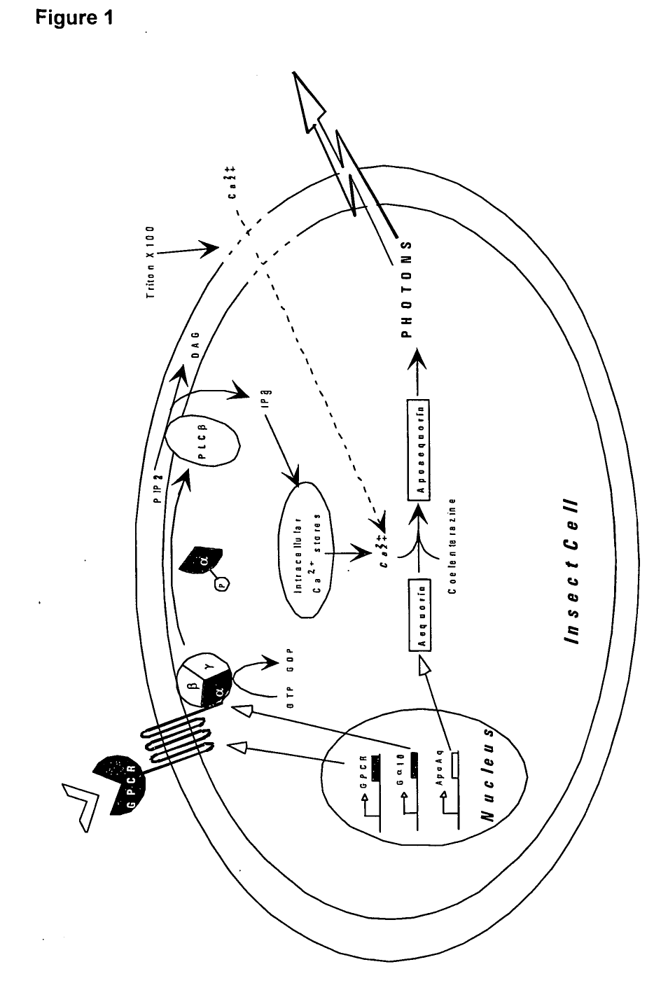

A mechanistic outline of an embodiment of an insect cell-based assay of the invention is shown in FIG. 1 for purposes of illustration (the actual mechanisms of action of various embodiments of the invention may be the same or different from the conceptual interactions illustrated in FIG. 1). The gene and protein components of the system include a human target GPCR, the human G-protein Galpha subunit Galpha₁₆⁷ and a Ca²⁺-sensitive bioluminescent reporter protein, aequorin⁸. As is disclosed herein, the human Galpha₁₆ subunit and endogenous insect G beta gamma subunits can apparently combine to form a functional Gq-type G protein, which is capable of interacting with endogenous insect signal transduction systems; including the insect homologue of the effector enzyme phospholipase-Cβ (PLCβ). In alternative embodiments, the use of human Galpha₁₆ may make the assays of the invention broadly applicable to a wide range of GPCRs, including human GPCRs.

In the putative signal transduction pathway of some embodiments of the invention, activated PLCβ cleaves insect membrane inositol phospholipids to release diacylglycerol (DAG) and inositol triphosphate (IP₃), which in turn mediates the release of the second messenger Ca²⁺ from the endoplasmic reticulum. The intracellular Ca²⁺ flux is detectable by co-expressing the Ca²⁺-sensitive photoprotein (apo)aequorin, which forms a bioluminescent complex when linked to its chromophore co-factor coelenterazine. In the presence of Ca²⁺, oxidation of bound coelenterazine leads to a transient flash of light¹⁰, detectable with a luminometer¹¹. Aequorin bioluminescence has the advantage that the background “noise” is extremely low, since cells do not spontaneously produce light; consequently, the assay is very sensitive and has a large dynamic range¹⁰. A number of luminometers suitable for automated bioluminescent screening are commercially available¹², and may be selected so as to be capable of quantitating this assay procedure.

In various embodiments, the invention provides genes having coding sequences that are transcribed and translated by host insect cell lines, so that the protein products preferably assemble in their correct cellular compartments where they interact with endogenous insect proteins involved in GPCR signal transduction pathways. In alternative embodiments, these genetically engineered insect tissue culture cell lines (“insect-based assay cell lines”) can be made using a wide variety of insect expression vector systems capable of establishing transiently- or permanently-transformed insect cell lines¹³⁻¹⁵. For example, cDNA's encoding human GPCRs, the human Galpha₁₆ G-protein subunit, and a bioluminescent reporter protein (aequorin) were transferred into the multiple cloning region of the vector p2Zop2F¹⁵, and amplified in an E. coli host strain under Zeocin™ selection. Selected recombinant vectors (designated p2Z2F-GPCR, -G16 and -Aq respectively) were purified and used for cell line co-transformations (typically, but not necessarily in a 1:1:1 ratio) as described in Hegedus et al, 1998¹⁶. Insect tissue culture cell lines used to create insect-based assay cell lines include (but are not limited to) the lepidopteran Sf9 and Hi5 cell lines, and the dipteran Kc1 and SL2 cell lines. Insect cell lines either transiently or permanently transformed with all three heterologous genes met the minimal requirements to behave as insect-based assay cell lines; that is, they responded to application of a receptor-specific ligand with a flash of light. Insect tissue culture cell lines either transiently or permanently transformed with the genes described above can be used for the assay, but permanently transformed cells may be preferred.

A wide variety of insect expression vectors may be used in various aspects of the invention to express recombinant nucleic acids in transformed insect cells. For example, International Patent Publication No. WO9844141 published on 8 Oct. 1998 and incorporated herein by reference discloses a variety of insect shuttle vectors, and methods of using such vectors, for stably transforming disparate insect cell lines to express heterologous proteins. The invention disclosed therein provides a transformed insect cell selection system based on resistance to the bleomycin/phleomycin family of antibiotics, including the antibiotic Zeocin. Efficient promoters derived from baculovirus immediate early promoters are disclosed for use in directing expression of heterologous proteins, including selectable markers, in transformed insect cells of the invention. A variety of promoters may be used to direct heterologous protein production in insect cells. The ACMNPV immediate-early (ie) promoters have been used successfully to express highly modified proteins in lepidopteran (Jarvis et al., Bio/Technology, 8: 950-955 (1990)), D. melanogaster (Morris and Miller, J. Virol., 66: 7397-7305 (1992)) as well as mosquito cells where levels were comparable to those directed by the heat-shock promoter under full induction (Shotkoski et al., FEBS Lett., 380: 257-262 (1996)). It has been shown that the ie1 and ie2 promoters derived from the OpMNPV genes function effectively in dipteran and lepidopteran cell lines and can be used to drive expression of heterologous genes (Hegedus et al., Gene 207:241-249 (1998)). A series of versatile expression vectors which use the OpMNPV ie2 promoter for constitutive heterologous protein expression in dipteran and lepidopteran insect cells or the Mtn promoter for inducible expression in dipteran cell lines have been described previously (Hegedus et al., Gene 207:241-249 (1998)). The compact shuttle vectors utilize a chimeric promoter to allow selection for Zeocin-resistance in both insect cells and E. coli. This greatly enhances their utility by facilitating gene manipulation and DNA insertion. With the above advantages in hand, expression vectors based on zeocin and puromycin resistance were developed in the lab (Hegedus et al., Gene 207:241-249 (1998)) and have been used to express a number of heterologous proteins in insect cell lines (Hegedus et al., Prot. Exp. Purif. 15:296-307(1999); ITP; Factor X). Commercial embodiments of vectors suitable for mediating expression of heterologous proteins may for example be available from Invitrogen Corporation (Carlsbad, Calif., U.S.A.), such as the InsectSelect™ System for Sf9 and Hi5 cells.

In one embodiment, the assay system of the invention was implemented using the human dopamine D1 (hDD1) GPCR, which is normally coupled to a Gs-type G protein. The insect-based assay cell lines consisted of lepidopteran Sf9 cells permanently transformed with the genes for the hDD1 receptor, Galpha₁₆, and aequorin. Experiments were conducted using either an LKB 1250 tube luminometer or a Labsystems Fluoroskan Ascent FL microplate luminometer equipped with three auto-injectors. Any instrument capable of quantitating luminescent light output in small volumes could be used for this assay. Selected insect-based assay cell lines were grown in ESF media, harvested and then re-suspended in fresh ESF at a density of 5×10⁵cells/ml. Cells were primed by adding the aequorin co-factor coelenterazine (or any derivatives thereof, e.g. coelenterazine cp) to a final concentration of 0.5 microM. Maximal reconstitution of the holoenzyme apo-aequorin (aequorin+coelenterazine) was obtained by incubating the cells for 1 hour, at room temperature (23-26° C.), in the dark, and with constant rocking. Following this treatment, insect-based assay cell lines give a consistent response to agonists over a period of 24 hours, which represents the “window” of stability during which they can be used. Insect-based assay cell lines can be used to identify agonists or antagonists by a variety of methodologies. Ligands can be injected into wells already containing assay cell lines, or assay cell lines can be injected into wells already containing ligands

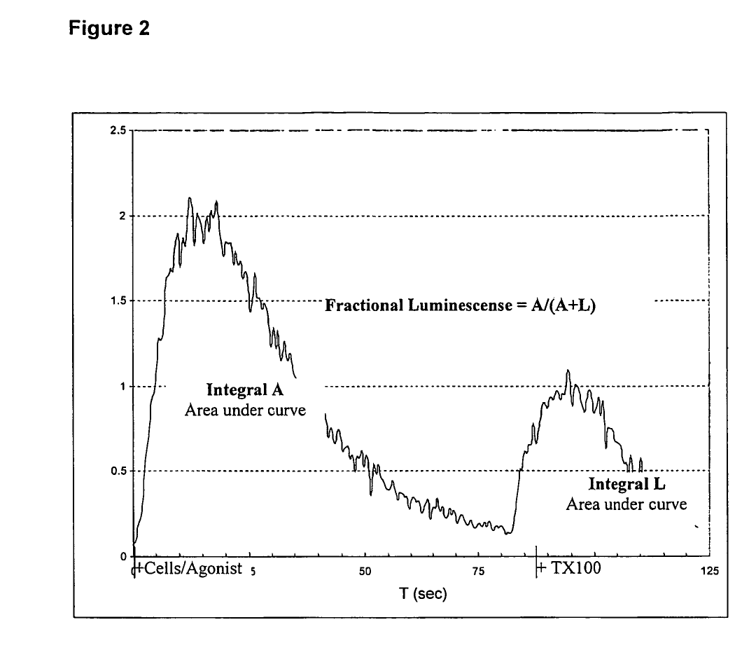

To assay GPCR agonists, a dilution series of the agonist in cell culture media (ESF) was arrayed in 10 microl aliquots in 96-well plate. Insect-based assay cell lines were maintained in a low volume, light-proof stirred flask. Injection of 50,000-100,000 cells in 100 microl of ESF media into an individual well initiated the experiment, and agonist-induced luminescense was be monitored for 75 seconds. A second injection of 50 microl of the detergent Triton X100 (TX100) in ESF was used to lyse the cells, and expose the totality of (apo)aequorin. Light output was monitored for 30 seconds (FIG. 2). Data analysis involved integration of the area under each peak (agonist, A and detergent, L{ysis}), then expressing the results as the ratio A/(A+L). This is the “fractional luminescense”, defined as that part of the total luminesense potential emitted in response to agonist challenge, thus self-correcting for any well-to-well variation in the number of injected cells (FIG. 2)).

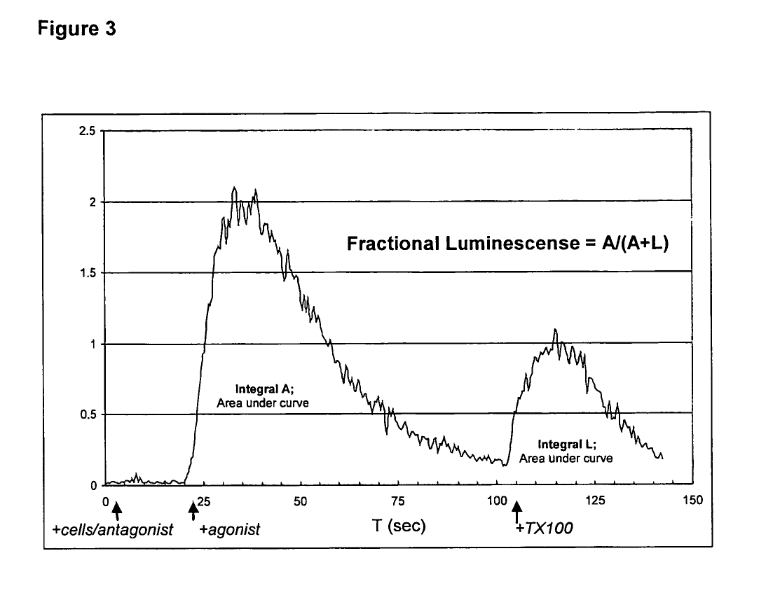

To assay GPCR antagonists required a modified protocol. The antagonist dilution series in ESF media was arrayed in 10 microl aliquots in a 96-well plate, 50,000-100,000 insect-based assay cells in 100 microl of ESF media was added to each well and the two were allowed to pre-incubate together for 30 seconds. The experiment was initiated by injecting an appropriate agonist, in a 10 microl volume of ESF. Light output was monitored for 75 seconds. A second injection of 50 microl of the detergent TX100 in ESF was used to lyse the cells, and expose the totality of (apo)aequorin. Light output was monitored for 30 seconds (FIG 3). Data analysis was performed as described above. An optional 10 second rocking step was added after each individual well was measured, to ensure that the insect-based assay cells arrayed in the plate stayed in suspension

Agonist and antagonist concentration-response curves were obtained by plotting graphs of A/(A+L) on the Y-axis versusLOG10 of ligand concentration (Molar) on the X-axis. Curve fitting was performed using the software package GraphPad Prism (GraphPad Software, Inc., San Diego, Calif., USA). The 3- or 4-Parameter Logistic equations were used for non-linear regression, and to acquire derived values (EC50 and Hill Slope).

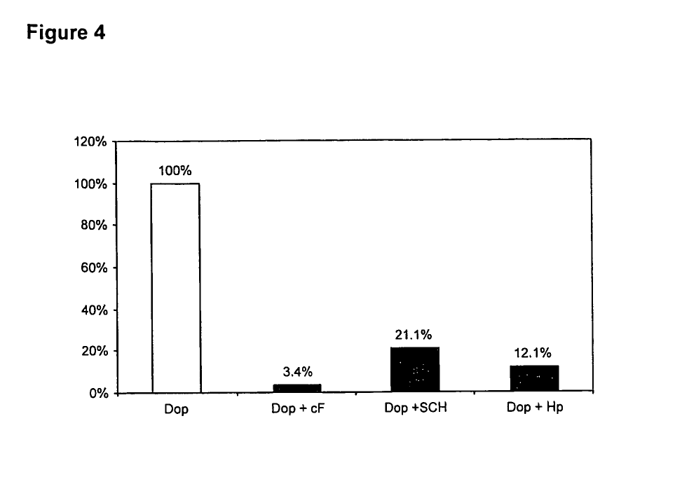

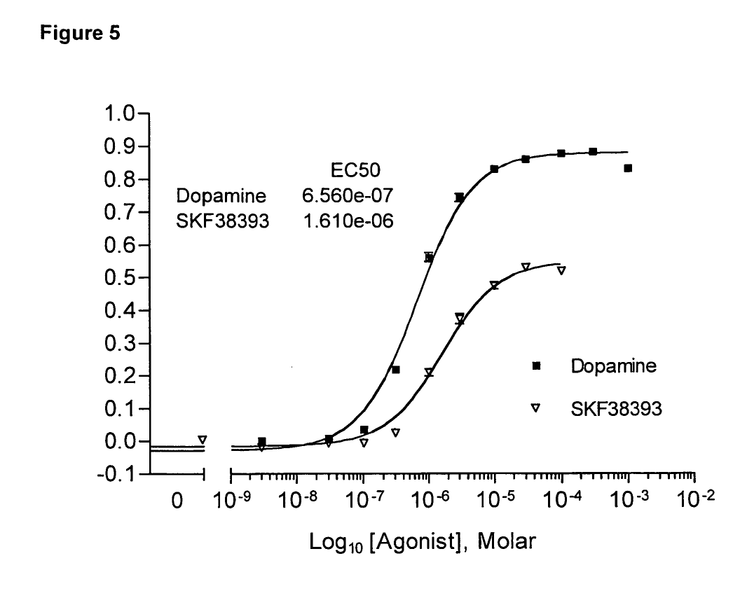

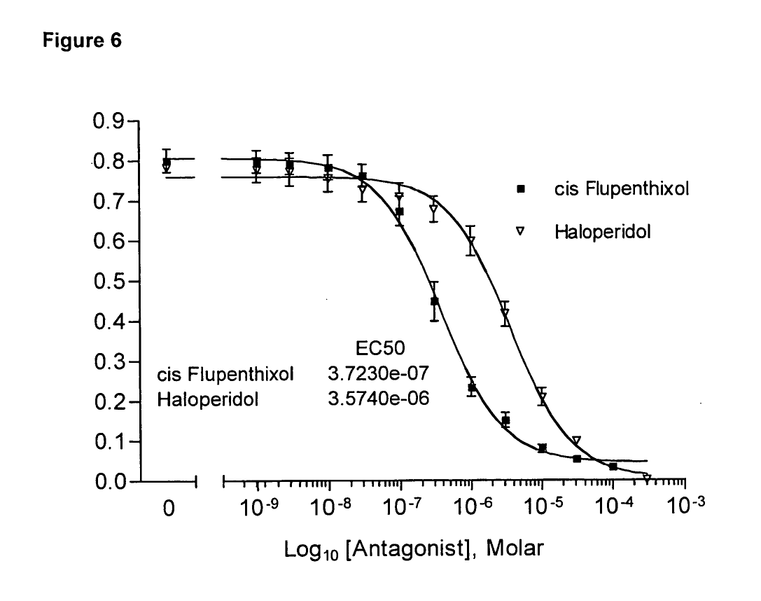

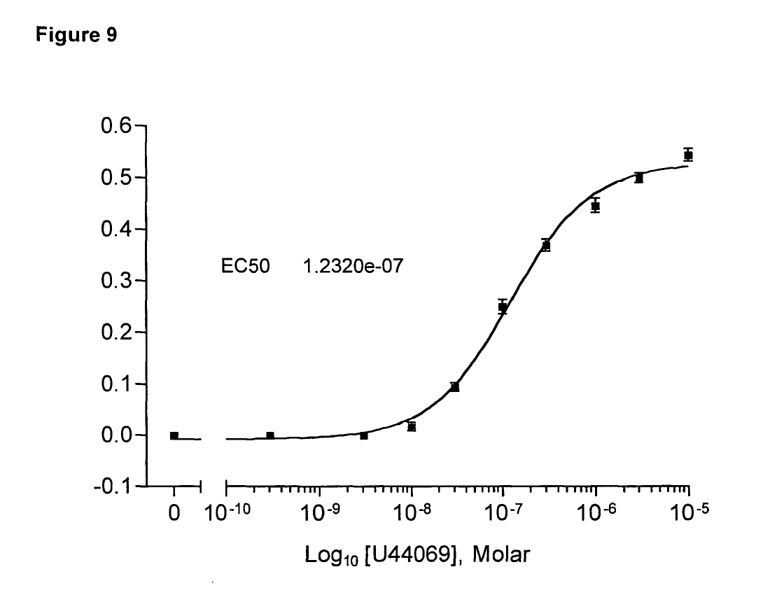

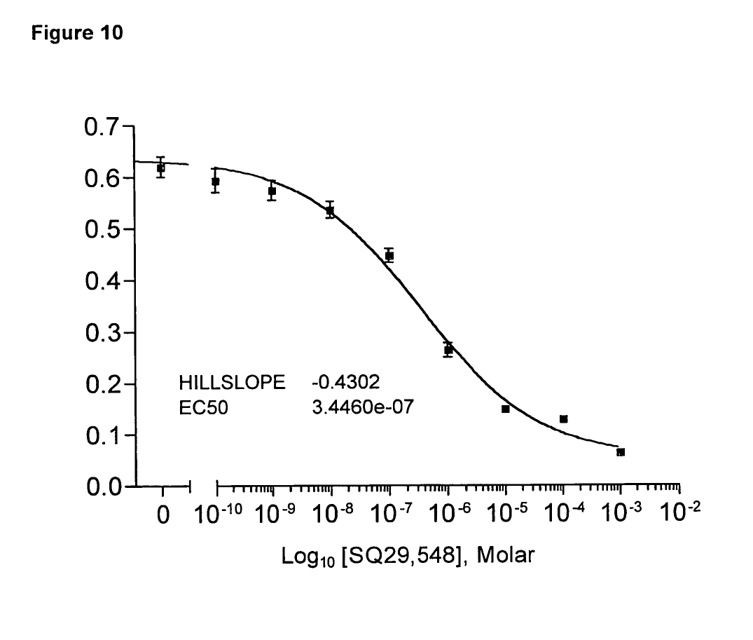

Insect assay cell lines containing the human Dopamine D1A GPCR (DADR assay cells) responded with a luminescent flash of light within seconds of being challenged with dopamine, the in vivo ligand for the receptor. They also showed a characteristic sigmoidal agonist concentration-activity curve when exposed to increasing concentrations of dopamine (FIG. 4). Similarly the Dopamine D1 receptor-specific agonist SKF38393 was able to trigger a luminescent response from DADR assay cells, but the D2-specific agonist LY-171555 was not. Dopamine agonist activation can be blocked by pre-incubation of the transformed cells with the dopamine receptor antagonist's cis-Flupenthixol (FIG. 4), SCH23390 and Haloperidol (FIG. 4). The insect-based cell assay can be used to construct antagonist concentration-activity curves (FIG. 6), as well as agonist concentration-activity curves (FIG. 5). These data collectively indicate that the DADR receptor retained its ligand specificity in insect assay cell lines. In control experiments performed with cells containing any two out of the three assay components, no luminescence was seen on the addition of dopamine. This indicates that all components of the assay as described are required for functional coupling of the DADR GPCR to apoaequorin luminescence, and demonstrates that endogenous insect G beta gamma sub-units are capable of functionally coupling to human Galpha₁₆. A similar series of experiments were performed with the human Thromboxane A2a (TXA2a) GPCR, and qualitatively similar results were obtained with TXA2a-specific agonists and antagonists (FIGS. 9 and 10). These data support the general utility of the core assay technology to multiple human GPCRs.

In addition to advantages that may be inherent to using insect cells in various aspects of the invention, there may be further advantages specific to the use of aequorin in systems of the invention. Firstly, in some embodiments, the optimal concentration of the aequorin cofactor coelenterazine required in insect cells, 0.5 microM in some embodiments, is substantially lower than that typically required in some mammalian cells (5 microM)¹⁷, which may result in a significant reduction in the cost of this assay reagent. Secondly, in some embodiments, insect assay cell lines may have a “window” of stable response to agonists of up to 24 hours, compared to reported response windows of only 5 hours in some mammalian cells¹⁷. Accordingly, in some embodiments, insect cells may be both cheaper and more flexible host cells for aequorin-mediated Ca²⁺ detection.

In some embodiments, the present invention has been used with a very closely related homologue of human Galpha₁₆, the murine Gq-type Galpha subunit Galpha₁₅⁹, which has been substituted for Galpha₁₆ in embodiments of the insect-cell based assay system of the invention without any significant difference in assay results. Various adaptations may also be made to the systems of the invention to increase the sensitivity of the insect-based cell assay. For example, co-expression of human PLCβ's¹⁸, and/or various combinations of human G beta and G_gamma subunits¹⁹ in insect-based assay cell lines may lead to increases in assay sensitivity. Co-expression in insect-based assay cells of the Drosophila ion channel trp²⁰ may also lead to an increase in assay sensitivity. In some embodiments, modification of a transfected aequorin cDNA clone to include a mitochondrial peptide targeting signal²¹ at the 5′ terminus increased the sensitivity of the assay by localizing the bioluminescent protein in the insect cell mitochondria.

In some embodiments, alternative detection technologies could be incorporated into the insect-based assay cell lines in place of the (apo)aequorin system. For example, any one of a number of Ca²⁺-sensitive flourescent dyes (for example, but not limited to, Fluo-3™ and Fluo-4™) could be employed to detect calcium flux, and the assay response measured with a fluorimeter rather than a luminometer. Alternatively, a GPCR-inducible promoter element linked to reporter gene (for example, but not limited to, green flourescent protein {GFP}, beta galactosidase, or chloramphenicol acetyltransferase{CAT}) could be inserted into the insect assay cell lines in place of the aequorin gene. In such embodiments, ligand-induced activation of the target GPCR would lead to activation of the GPCR-inducible promoter and production of the reporter protein, which can be quantitated. For example, the presence of GFP can be monitored by using a fluorimeter or by using a fluorescence-activated-cell-sorting (FACS) machine. In alternative embodiments, various calcium and cAMP-sensitive gene reporter systems may be used, wherein a calcium flux or cAMP modulation leads to a detectable signal through alteration of expression of a reporter gene.

In alternative embodiments, the insect cell-based assay of the invention can be constructed in a wide range of different insect tissue culture cell lines, including (but not limited to) lepidopteran lines (Sf9 and Hi5) and dipteran lines (SL2 and Kc1), which may be advantageous to exploit differences between insect cell lines (for example, but not limited to, differences in protein post-translational modifications such as glycosylation, myristoylation, palmitoylation, phosphorylation) which may enable or enhance the functioning of heterologous protein assay components such as target GPCRs or G protein subunits.

In various aspects, the invention may utilize a variety of G alpha proteins, including Gαolf, Gα12, Gαo1, Gα15, Gαs, Gα13, Gαo2, Gα16, Gαi1, Gα11, Gαi2, Gαq, Gαi3, Gα14, Gαz. It is well known in the art that some modifications and changes can be made in the structure of a polypeptide without substantially altering the biological function of that peptide, to obtain a biologically equivalent polypeptide. In one aspect of the invention, G alpha proteins used in the invention may differ from a portion of the corresponding native sequence by conservative amino acid substitutions. As used herein, the term “conserved amino acid substitutions” refers to the substitution of one amino acid for another at a given location in the peptide, where the substitution can be made without loss of function. In making such changes, substitutions of like amino acid residues can be made, for example, on the basis of relative similarity of side-chain substituents, for example, their size, charge, hydrophobicity, hydrophilicity, and the like, and such substitutions may be assayed for their effect on the function of the peptide by routine testing. In some embodiments, conserved amino acid substitutions may be made where an amino acid residue is substituted for another having a similar hydrophilicity value (e.g., within a value of plus or minus 2.0), where the following hydrophilicity values are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gln (+0.2); Gly (0); Pro (−0.5); Thr (−0.4); Ala (−0.5); His (−0.5); Cys (−1.0); Met (−1.3); Val (−1.5); Leu (−1.8); Ile (−1.8); Tyr (−2.3); Phe (−2.5); and Trp (−3.4). In alternative embodiments, conserved amino acid substitutions may be made where an amino acid residue is substituted for another having a similar hydropathic index (e.g., within a value of plus or minus 2.0). In such embodiments, each amino acid residue may be assigned a hydropathic index on the basis of its hydrophobicity and charge characteristics, as follows: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gin (−3.5); Asp (−3.5); Asn (−3.5); Lys (−3.9); and Arg (−4.5). In alternative embodiments, conserved amino acid substitutions may be made where an amino acid residue is substituted for another in the same class, where the amino acids are divided into non-polar, acidic, basic and neutral classes, as follows: non-polar: Ala, Val, Leu, lie, Phe, Trp, Pro, Met; acidic: Asp, Glu; basic: Lys, Arg, His; neutral: Gly, Ser, Thr, Cys, Asn, Gln, Tyr.

Various aspects of the present invention encompass nucleic acid or amino acid sequences that are homologous to other sequences, such as G alpha proteins that are homologous to known G alpha proteins. As the term is used herein, an amino acid or nucleic acid sequence is “homologous” to another sequence if the two sequences are substantially identical and the functional activity of the sequences is conserved (for example, both sequences function as or encode a selected enzyme or promoter function; as used herein, the term ‘homologous’ does not infer evolutionary relatedness). Nucleic acid sequences may also be homologous if they encode substantially identical amino acid sequences, even if the nucleic acid sequences are not themselves substantially identical, a circumstance that may for example arise as a result of the degeneracy of the genetic code.

Two nucleic acid or protein sequences are considered substantially identical if, when optimally aligned, they share at least about 25% sequence identity in protein domains essential for conserved function. In alternative embodiments, sequence identity may for example be at least 50%, 70%, 75%, 90% or 95%. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence alignment may also be carried out using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nim.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST programs may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (which may be changed in alternative embodiments to 1 or 0.1 or 0.01 or 0.001 or 0.0001; although E values much higher than 0.1 may not identify functionally similar sequences, it is useful to examine hits with lower significance, E values between 0.1 and 10, for short regions of similarity), M=5, N=4, for nucleic acids a comparison of both strands. For protein comparisons, BLASTP may be used with defaults as follows: G=11 (cost to open a gap); E=1 (cost to extend a gap); E=10 (expectation value, at this setting, 10 hits with scores equal to or better than the defined alignment score, S, are expected to occur by chance in a database of the same size as the one being searched; the E value can be increased or decreased to alter the stringency of the search.); and W=3 (word size, default is 11 for BLASTN, 3 for other blast programs). The BLOSUM matrix assigns a probability score for each position in an alignment that is based on the frequency with which that substitution is known to occur among consensus blocks within related proteins. The BLOSUM62 (gap existence cost=11; per residue gap cost=1; lambda ratio=0.85) substitution matrix is used by default in BLAST 2.0. A variety of other matrices may be used as alternatives to BLOSUM62, including: PAM30 (9,1,0.87); PAM70 (10,1,0.87) BLOSUM80 (10,1,0.87); BLOSUM62 (11,1,0.82) and BLOSUM45 (14,2,0.87). One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially identical is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, stringent conditions are selected to be about 5° C. lower than the thermal melting point for the specific sequence at a defined ionic strength and pH.

GPCRs for use in various aspects of the invention may be identified by homology to known GPCRs. For example, Class A Rhodopsin like GPCRs may be identified by homology to known GPCRs such as the Rhodopsin Vertebrate type 1, type 2, type 3, type 4 or type 5 receptors. For example, the following is a list of a number of human GPCR sequences available in the Swiss-Prot and TrEMBL databases, providing ID, accession, gene name and putative GPCR family:

OPN4_HUMAN (Q9UHM6) OPN4 OR MOP Rhodopsin Other

OPSB_HUMAN (PO₃₉₉₉) BCP Rhodopsin Vertebrate type 3

OPSD_HUMAN (P08100) RHO OR OPN2 Rhodopsin Vertebrate type 1

OPSG_HUMAN (PO₄₀₀₁) GCP Rhodopsin Vertebrate type 2

OPSR_HUMAN (PO₄₀₀₀) RCP Rhodopsin Vertebrate type 2

OPSX_HUMAN (O14718) RRH Rhodopsin Other

5H1A_HUMAN (P08908) HTRIA Serotonin Vertebrate type 1

5H7_HUMAN (P34969) HTR7 Serotonin Vertebrate type 7

A1AA_HUMAN (P35348) ADRA1A OR ADRA1C Alpha Adrenoceptors type 1

A2AD_HUMAN (P35369) none Alpha Adrenoceptors type 2

AA1R_HUMAN (P30542) ADORA1 Adenosine type 1

ACM1_HUMAN (P11229) CHRM1 Acetylcholine Vertebrate type 1

AG22_HUMAN (P50052) AGTR2 Angiotensin type 2

APJ_HUMAN (P35414) AGTRL1 OR APJ APJ like

BAI1_HUMAN (O14514) BAI1 Brain-specific angiogenesis inhibitor (BAI)

BRB1_HUMAN (P46663) BDKRB1 OR BRADYB1 Bradykinin

BRS3_HUMAN (P32247) BRS3 Bombesin

C3X1_HUMAN (P49238) CX3CR1OR GPR13 C-X3-C Chemokine

C5AR_HUMAN (P21730) C5R1OR C5AR C5a anaphylatoxin

CALR_HUMAN (P30988) CALCR Calcitonin

CASR_HUMAN (P41180) CASR OR PCAR1 Extracellular calcium-sensing

CB1R_HUMAN (P21554) CNR1 OR CNR Cannabis

CCKR_HUMAN (P32238) CCKAR OR CCKRA CCK type A

CCR3_HUMAN (P49682) CXCR3OR GPR9 C-X-C Chemokine type 3

CKR2_HUMAN (P41597) CCR2OR CMKBR2 C-C Chemokine type 2

D2DR_HUMAN (P14416) DRD2 Dopamine Vertebrate type 2

EDG1_HUMAN (P21453) EDG1 Lysosphingolipid & LPA (EDG)

EMR1_HUMAN (Q14246) EMR1 EMR1

ET1R_HUMAN (P25101) EDNRA OR ETRA Endothelin

FML2_HUMAN (P25089) FPRL20R FPRH1 Finet-leu-phe

GALR_HUMAN (P47211) GALR1 OR GALNR1OR GALNR Galanin

GLP1_HUMAN (P43220) GLP1R Glucagon

GP40_HUMAN (014842) GPR40 GP40 like

HH1R_HUMAN (P35367) HRH1 Histamine type 1

MC3R_HUMAN (P41968) MC3R Melanocortin hormone

MGR1_HUMAN (Q13255) GRM10R MGLUR1 Metabotropic glutamate group 1

ML1X_HUMAN (Q13585) GPR50 Melatonin

O1D4_HUMAN (P47884) OR1 D4 Olfactory type 1

Q13675 (Q13675) none Alpha Adrenoceptors type 1

In the context of the present invention, a moiety such as a nucleic acid or protein is “heterologous” if it is present by virtue of human intervention in a cell in which it is not naturally present, irrespective of whether the moiety is derived from the same species or a difference species. A cell into which has been introduced a foreign (heterologous) nucleic acid, is considered “transformed”, “transfected” or “transgenic” because it contains the heterologous nucleic acid introduced by human intervention. Progeny of the cell that is initially transformed with a recombinant nucleic acid construct is also considered “transformed”, “transfected” or “transgenic”. The invention provides vectors, such as vectors for transforming insect cells. The term “vector” in reference to nucleic acid molecule generally refers to a molecule that may be used to transfer a nucleic acid segment(s) from one cell to another. A recombinant nucleic acid is a nucleic acid molecule that has been recombined or altered by human intervention using techniques of molecular biology. Generally, a recombinant nucleic acid comprises nucleic acid segments from different sources ligated together, or a nucleic acid segment that is removed from the adjoining segments with which it is naturally joined.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and Figures.

### EXAMPLES

Materials and Methods

Insect Cell Lines and Growth Conditions

Sf9 (Spodoptera frugiperda) and Hi5 (Trichoplusia ni) cell lines (Invitrogen, USA) were used in these studies. The cell lines were maintained on T25 flasks at 26° C. in ESF921 media (Expression Systems LLC, CA, USA), which is serum and protein free. When necessary, cell lines were scaled up in 50 ml spinners operating at 80 rpm and 26° C.

Preparation of DNA for Transfections

DNA was purified either by CsCl gradients or Qiagen columns (Qiagen, Germany). DNA was quantified using a spectrophotometer and purity assessed using an OD260/280 ratio. A ratio between 1.8 and 2.0 was required. DNA concentration and conformation was confirmed by agarose gel electrophoresis.

Transfection of Insect Cell Lines with DNA Vectors

Typically 1 to 2×10⁶ cells were plated in 1 ml of minimal Grace's (Gibco-BRL) onto one well (30 mm) of a 6 well culture plate and the cells allowed to attach. A total of one μg of DNA was mixed in 0.5 ml of Grace's and separately 10 μl of CellFectin (Gibco-BRL) mixed in another 0.5 ml of Grace's. These two 0.5 ml aliquotes were then combined, mixed and allowed to incubate at room temperature. After 30 minutes, the Grace's was removed from the cells and the cells then overlaid with the DNA/CellFectin solution. The plate was rocked once every hour and after 4 hours one ml of ESF921 was added, the plate sealed with making tape and incubated for 48 hr at 26° C.

Creation of Stable Insect Cell Lines

After transfection of the cell lines with DNA as described above, Zeocin or puromycin resistant cell populations were selected for by addition of either 1 mg/ml Zeocin or 2-microg/ml puromycin. Within 3-4 weeks of transformation resistant populations of cells were generated. These were maintained as polyclonal cultures under selection. Once stable cell lines were obtained, master stocks were made and kept in liquid nitrogen. This involved harvesting mid-log phase cells, adding 7.5% DMSO and cooling to −70° C. at a slow rate (1° C./min). Cells were then transferred to a liquid nitrogen tank.

Construction of Heterologous Expression Vectors for Use in Insect Cells.

The insect expression vectors p2Zop2F and variants previously described (Hegedus et al., Gene 207:241-249(1998)) were used throughout these studies except as noted.

p2Zop2SK

The SK and KS primers from pBSKSII were used to amplify the multiple cloning site of pBSKSII which was then cloned into the HindIII (blunted with Klenow and dNTP's) EcoRV site of p2ZOp2F. The resulting vector was named p2ZOp2SK

pA2E

A BspHI fragment from pBKSII containing the ampicillin resistance gene was ligated to a BspHI fragment of p2Zop2E containing the expression cassette. The resulting vector, pA2E, provided ampicillin resistance for selection in bacteria, and the ie-2 promoter to direct expression of heterologous gene products in bacteria and insect cells

Dopamine D1 (D1) Receptor

A 1,970 bp EcoRI-XbaI fragment containing the human Dopamine D1 receptor cDNA (pSP65DD1: Nature 74:80(1990)) was cloned into the EcoRI-XbaI site of p2ZoP2F to create D1:p2ZOp2F. To eliminate 300 bp of sequence upstream of the ATG start of translation, a PCR was performed with the primers D1F (ATGAGGACTCTGMCMCTCTGC) and D1B (CATGGCAGAGTTGTTCAGAGTCCTCAT) which amplified the first 300 bp of the gene. This PCR product was cut with BglII and the resulting 200 bp fragment inserted into the blunted EcoRI (with klenow and dNTP's)—BglII site of D1:p2Zop2F creating D1-short:p2Zop2F. The gene was sequenced to ensure that the reading frame and sequence of the gene was intact. Sequence analysis showed the correct gene sequence was amplified as reported in Genbank ACCESSION XM_{—}003966.

Thromboxane A2 (TXA2) Receptor

A 1200 bp BamHI-HindIII fragment from pCMX-TXA2 (Nature (1991)349:617) was cloned into the BamHI-HindIII site of p2Z2SK. To remove a false ATG start of translation, the construct was then cleaved with BspEI-XbaI, blunted with Klenow and dNTP's and religated. The resulting vector TXA2:p2Zop2F was sequenced to confirm proper sequence and reading frame for the ORF. Sequence analysis showed the correct gene sequence was amplified as reported in Genbank ACCESSION XM_{—}047632.

Serotonin 5-HT2A (5-HT2A) Receptor

A 1600 bp NcoI (blunted with klenow and dNTPs)-XbaI fragment from pcDNA3-5HT2A containing the Serotonin 2A receptor, was cloned into the HindIII (blunted with klenow and dNTP's)-XbaI site of p2ZOp2F. The resulting vector 5-HT2A:p2Zop2F was sequenced to confirm proper sequence and reading frame for the ORF. Sequence analysis showed the correct gene sequence was amplified as reported in Genbank ACCESSION S71229.

Adrenergic, beta-2 (ADBR2) Receptor

The ADBR2 gene was amplified from human genomic DNA using the primers ADBR2F(5′-AAGCTTCAACATGGGGCMCCCGGGAACGGCAG-3′) and ADBR2R (5′-TCTAGATTTACAGCAGTGAGTCATTTGTACTAC-3′) and Pfu polymerase. A 1250 bp fragment was cloned into the EcoRV site of PBKSII and the resulting insert sequenced using the T3 and T7 primers. Sequence analysis showed the correct gene sequence was amplified as reported in Genbank ACCESSION NM_{—}000024. The insert was cleaved with HindIII-XbaI and placed into the HindIII-XbaI site of p2ZOp2F to yield ADBR2,p2ZOp2F.

M1 Muscarinic Acetylcholine (CHRM1) Receptor

The CHRM1 gene was amplified from human genomic DNA using the primers CHRM1F(5′-MGCTTMCATGAACACTTCAGCCCCACCTGC-3′) and CHRM1R (5′-TCTAGATTTAGCATTGGCGGGAGGGAGTGCGG-3′) and Pfu polymerase. A 1400 bp fragment was cloned into the EcoRV site of pBKSII and the resulting insert sequenced using the T3 and T7 primers. Sequence analysis showed the correct sequence was amplified as reported in Genbank ACCESSION NM_{—}000738. The insert was cleaved with HindIII-XbaI and placed into the HindIII-XbaI site of p2ZOp2F to yield CHRM1:p2ZOp2F.

5-Hydroxytryptamine Receptor 1A (5HT1A)

The 5HT1A gene was amplified from human genomic DNA using the primers 5HT1AF(5′-AAGCTTMCATGGATGTGCTCAGCGCTGGTCAG-3′) and 5HT1AR (5′-TCTAGATTTACTGGCGGCAGMCTTACACTTA-3′) and Pfu polymerase. A 1260 bp fragment was cloned into the EcoRV site of pBKSII and the resulting insert sequenced using the T3 and T7 primers. Sequence analysis showed the correct sequence was amplified as reported in Genbank ACCESSION NM_{—}000524. The insert was cleaved with HindIII-XbaI and placed into the HindIII-XbaI site of p2ZOp2F to yield 5HT1A:p2ZOp2F.

Histamine H1 Receptor (HRH1)

The HRH1 gene was amplified from human genomic DNA using the primers HRH1F(5′-AAGCTTACMTGAGCCTCCCCMTTCCTCCTG-3′) and HRH1R (5′-TCTAGATTTAGGAGCGMTATGCAGMTTC-3′) and Pfu polymerase. A 1450 bp fragment was cloned into the EcoRV site of pBKSII and the resulting insert sequenced using the T3 and T7 primers. Sequence analysis showed the correct sequence was amplified as reported in Genbank ACCESSION NM_{—}000861. The insert was cleaved with HindIII-XbaI and placed into the HindIII-XbaI site of p2ZOp2F to yield HRHI:p2ZOp2F.

Aequorin

An 800 bp SalI-EcoRI fragment from pDP5 (Anal. Biochem (1993) 209:343-347) containing the Aequroin gene was placed into the XhoI-EcoRI site of p2Zop2F yielding the plasmid Aq:p2Zop2F.

G Protein Alpha Subunit Alpha 15

A 1,353 bp NcoI (blunted with Klenow and dNTP's)—XbaI fragment containing the Galphal5 cDNA from pCISGalphal5 (PNAS(1991) 88:10049-10053) was cloned into the HindIII(blunted with Klenow and dNTP's)-XbaI site of p2Zop2F. Sequencing confirmed the proper reading frame of the ORF.

G Protein Alpha Subunit Alpha 16

A 1500 bp XhoI-SacI fragment from pCln containing the human Galphal6 cDNA was insert into the XhoI-SacI site of PBKSII (Construct A). To eliminate 5′ untranslated sequences upstream of the ATG start of protein translation and a false ATG start site, a 400 bp Sacll-XbaI fragment from the above construct was ligated into the SacII-XbaI site of pBKSII (Construct B). Construct B was cleaved with NcoI and SalI, releasing a small 200 bp fragment containing the false ATG start site, and the vector backbone religated (Construct C). A 1.0 kb SacII-SacI fragment from Construct A was ligated to a SacII:SacI cleaved Construct C to yield Construct D. Construct D contains the entire Galphal6 cDNA minus the 5′ upstream sequences. Sequence analysis showed the correct sequence was present as reported in Genbank ACCESSION M63904 The G alpha 16 cDNA fragment was released from Construct D by cleavage with KpnI-SacI and cloned into the KpnI-SacI sites of p2Zop2F and pA2E yielding the constructs G16:p2Zop2F and G16pA2E respectively.

Insect Cell Line Protocols

In some embodiments, the insect-based assay cell lines consisted of lepidopteran Sf9 cells permanently transformed with the cDNA's for one or more human GPCRs, Galpha₁₆, and aequorin. Experiments were conducted using either an LKB 1250 tube luminometer or a Labsystems Flouroscan Ascent FL microplate luminometer equipped with three auto-injectors. Any instrument capable of quantitating luminescent light output in small volumes could be used for this assay. Selected insect-based assay cell lines were grown in ESF media, harvested and then re-suspended in fresh ESF at a density of 5×10⁵cells/ml. Cells were primed by adding the aequorin co-factor coelenterazine (or any derivatives thereof, e.g. coelenterazine cp) to a final concentration of 0.5 microM. Maximal reconstitution of the holoenzyme apoaequorin (aequorin+coelenterazine) was obtained by incubating the cells for 1 hour, at room temperature (23-26° C.), in the dark, and with constant rocking. Following this treatment, insect-based assay cell lines give a consistent response to agonists over a period of 24 hours, which represents the “window” of stability during which they can be used. Insect-based assay cell lines can be used to identify agonists or antagonists by a variety of methodologies. Ligands can be injected into wells already containing assay cell lines, or assay cell lines can be injected into wells already containing ligands

GPCR Agonist and Antagonist Assay Protocols

To assay GPCR agonists, a dilution series of the agonist in ESF media was arrayed in 10 microl aliquots in 96-well plate. Insect-based assay cell lines were maintained in a low volume, light-proof stirred flask. Injection of 50,000-100,000 cells in 100 microl of ESF media into an individual well initiated the experiment, and agonist-induced luminescense was be monitored for 70 seconds. A second injection of 50 microl of the detergent TX100 in ESF was used to lyse the cells, and expose the totality of (apo)aequorin. Light output was monitored for 30 seconds (FIG. 2). Data analysis involved integration of the area under each peak (agonist, A and detergent, L{ysis}), then expressing the results as the ratio A/(A+L). This is the “fractional luminescense”, defined as that part of the total luminesense potential emitted in response to agonist challenge, thus self-correcting for any well-to-well variation in the number of injected cells.

To assay GPCR antagonists, a modified protocol may be used. The antagonist dilution series in ESF media was arrayed in 10 microl aliquots in a 96-well plate, 50,000-100,000 insect-based assay cells in 100 microl of ESF media was added to each well and the two were allowed to pre-incubate together for 30 seconds. The experiment was initiated by injectingan appropriate agonist in a 10 microl volume of ESF. Light output was monitored for 70 seconds. A second injection of 50 microl of the detergent TX100 in ESF was used to lyse the cells, and expose the totality of (apo)aequorin. Light output was monitored for 30 seconds (FIG. 3). Data analysis was performed as described above

Agonist and antagonist concentration-response curves were obtained by plotting graphs of A/(A+L) on the Y-axis versusLOG10 of ligand concentration (Molar) on the X-axis.Curve fitting was performed using the software package GraphPad Prism (GraphPad Software, Inc., San Diego, Calif., USA). The 3- or 4-Parameter Logistic equations were used for non-linear regression, and to acquire derived values (EC50 and Hill Slope).

### EXAMPLE ONE

### Human Dopamine DIA GPCR

The cDNA for the human D1A dopamine receptor (SwissProt; DADR_HUMAN; AC P21728) was cloned into the insect expression vector p2Zop2F as described. The Galpha₁₆, and aequorin expression constructs were as described.

DADR insect-based assay cell lines were created by simultaneous cotransfection of Sf9 insect cells with all three expression constructs as described herein. Both transiently-transfected and stable, zeocin-selected permanently transformed assay cell lines were used for the experiments described below. The human Dopamine D1A GPCR is coupled in vivo to a Gs-type G-protein, which causes the activation of the effector enzyme adenyly cyclase leading to an increase in the intracellular concentration of the second messenger cAMP.

Stable, zeocin-selected permanently transformed DADR assay cell lines showed a characteristic sigmoidal agonist concentration-activity curve when exposed to increasing concentrations of the endogenous agonist dopamine, or the DADR-specific artificial agonist SKF38393 (FIG. 5)

Dopamine agonist activity can be blocked by pre-incubation of the DADR assay cell line with the dopamine receptor antagonists cis-Flupenthixol or Haloperidol (FIGS. 4 and 6). Characteristic sigmoidal antagonist concentration-activity curves are obtained by varying antagonist concentrations against a fixed concentration of agonist (dopamine at 100 microM)

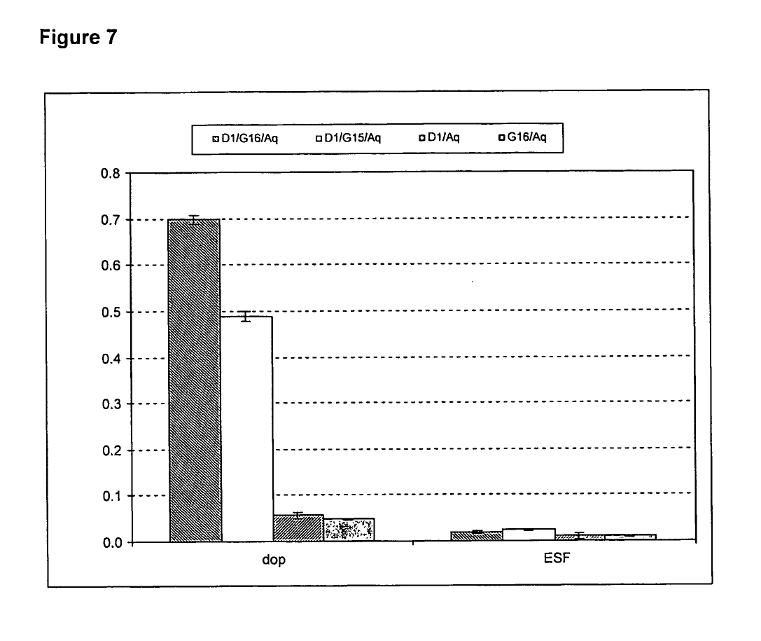

These data collectively indicate that the insect assay system is able to couple the human Dopamine D1A GPCR to a Gq-type G-protein containing human Galpha₁₆ and insect G beta gamma sub-units. Ligand-induced activation of this Gq-type subunit leads to activation of the endogenous insect effector protein phospholipase Cbeta, and an increase in intracelluar calcium concentration which can be detected by the calcium-sensitive photoprotein aequorin. In control experiments performed with cells transiently transfected any two out of the three assay components, no significant luminescence was seen on the addition of dopamine (FIG. 7). This indicates that all components of the assay as described are required for functional coupling of the human Dopamine D1A GPCR to apoaequorin luminescence, and demonstrates that endogenous insect G beta gamma sub-units are capable of functionally coupling to human Galpha₁₆. Human Galpha₁₆ is closely related to murine Galpha₁₅, which capable of substituting for Galpha₁₆ in DADR assay cell lines (FIG. 7).

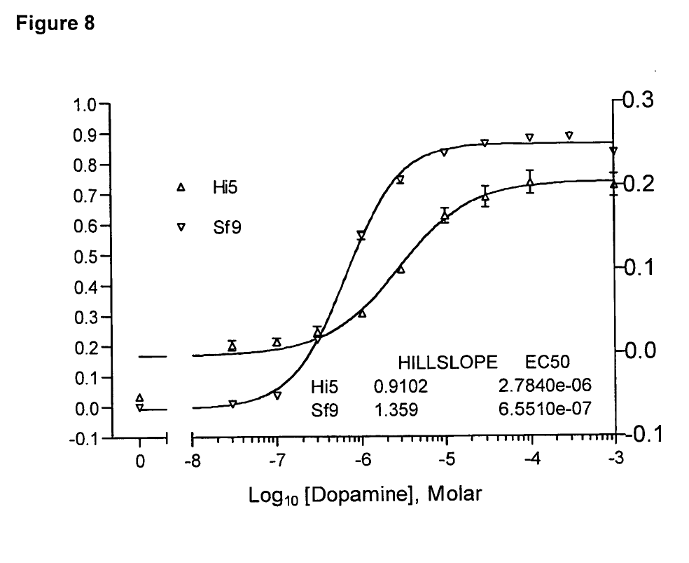

In addition to the Sf9 insect cell line, the Hi5 insect cell line (derived from Trichoplusia ni) is also able to support functional assays for human GPCRs. When co-transfected with the Dopamine D1A for Galpha₁₆ and aequorin expression constructs as described above, Hi5-DADR assay cell lines show the characteristic sigmoidal agonist concentration-activity curve when exposed to increasing concentrations of the endogenous agonist dopamine. The important parameters of the curve (Hill Slope and EC50) are very similar to those obtained from Sf9-DADR assay cell lines (FIG. 8). This indicates that a variety of insect cell lines are likely to be suitable for use as host cells for functional assays of human GPCRs. It is likely that subtle differences between insect cell lines (for example, in their abilities to perform post-translational modifications, their endogenous G-proteins or signal transduction effector proteins) can be usefully exploited to maximise the potential of insect cell-based assays for human GPCRs.

### EXAMPLE TWO

### Human Thromboxane A2 GPCR

The cDNA for the human Thromboxane A2 receptor (SwissProt; TA2R_HUMAN; AC P21731;) was cloned into the insect expression vector p2Zop2F as described herein. The Galpha₁₆, and aequorin expression constructs were as described herein.

TA2R insect-based assay cell lines were created by simultaneous cotransfection of Sf9 insect cells with all three expression constructs as described herein. Transiently-transfected assay cell lines were used for the experiments described below. The human Thromboxane A2 GPCR is coupled in vivo to a Gq-type G-protein, which causes the activation of the effector enzyme phospholipase Cbeta, leading to an increase in the intracellular concentration of the second messenger calcium.

Transiently-transfected TA2R assay cell lines showed a characteristic sigmoidal agonist concentration-activity curve when exposed to increasing concentrations of the agonist U44069 (FIG. 9), thus demonstrating functional coupling of the human Thromboxane A2 receptor to endogenous insect phospholipase Cbeta.

U44069 agonist activity can be blocked by pre-incubation of the TA2R assay cell line with the thromboxane A2 receptor-specific antagonist SQ29,548 (FIG. 10). Characteristic sigmoidal antagonist concentration-activity curves are obtained by varying antagonist concentrations against a fixed concentration of agonist (U44069 at 10 microM).

These experiments demonstrate that the human Thromboxane A2 GPCR is functionally expressed in insect cells, and able to signal through a hybrid G-protein consisting of human Galpha₁₆ and insect G beta gamma subunits. The net result of G protein activation is activation of endogenous insect phospholipase Cbeta, which in turn leads to an increase in intracellular calcium.

### EXAMPLE THREE

### Human Histamine H1 GPCR

The cDNA for the human Histamine H1 receptor (SwissProt; HH1 R_HUMAN; AC P35367;) was cloned into the insect expression vector p2Zop2F as described herein. The Galpha₁₆, and aequorin expression constructs were as described herein.

HH1 R insect-based assay cell lines were created by simultaneous cotransfection of Sf9 insect cells with all three expression constructs as described herein. Transiently-transfected assay cell lines were used for the experiments described below. The human HH1R GPCR is coupled in vivo to a Gs-type G-protein, which causes the activation of the effector enzyme adenylyl cyclase, leading to an increase in the intracellular concentration of the second messenger cAMP.

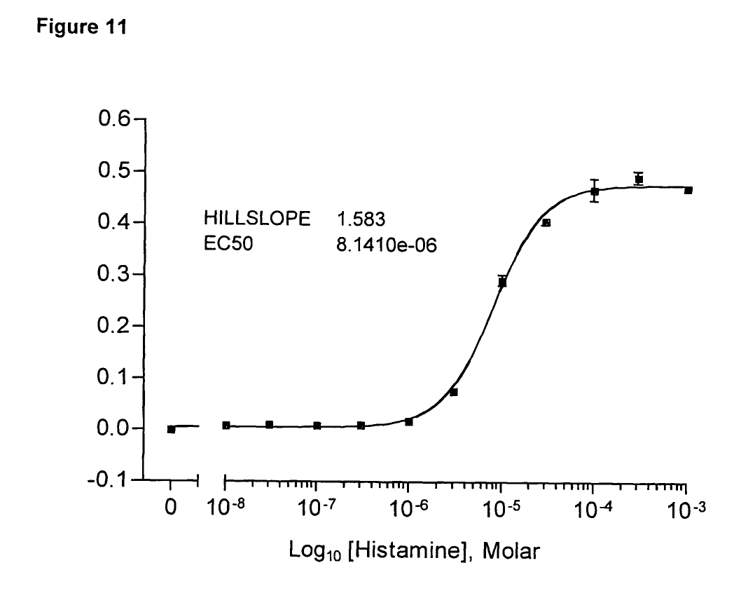

Transiently-transfected HH1 R assay cell lines showed a characteristic sigmoidal agonist concentration-activity curve when exposed to increasing concentrations of the endogenous agonist histamine (FIG. 11), thus demonstrating functional coupling of the human HH1R receptor to endogenous insect phospholipase Cbeta.

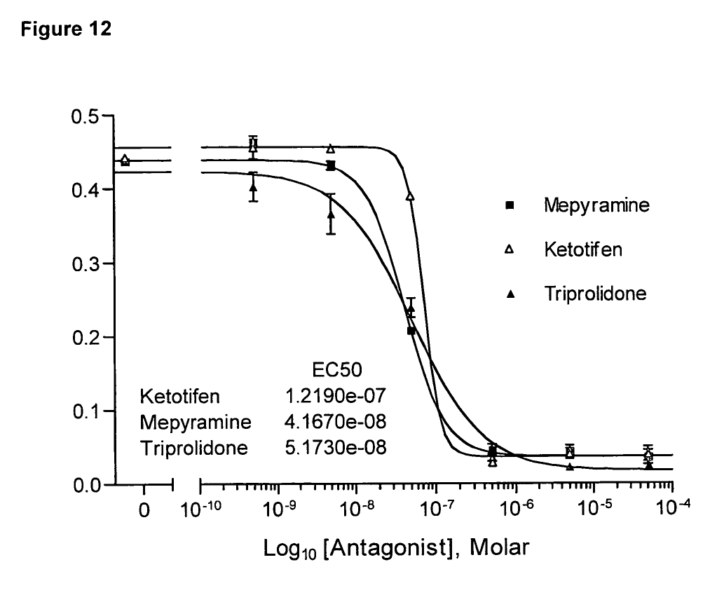

Histamine agonist activity can be blocked by pre-incubation of the 5H1A assay cell line with any of the Histamine H1-selective antagonists ketotifen fumarate, mepyramine maleate or triprolidone hydrochloride (FIG. 12). Characteristic sigmoidal antagonist concentration-activity curves are obtained by varying antagonist concentrations against a fixed concentration of agonist (histamine at 100 microM).

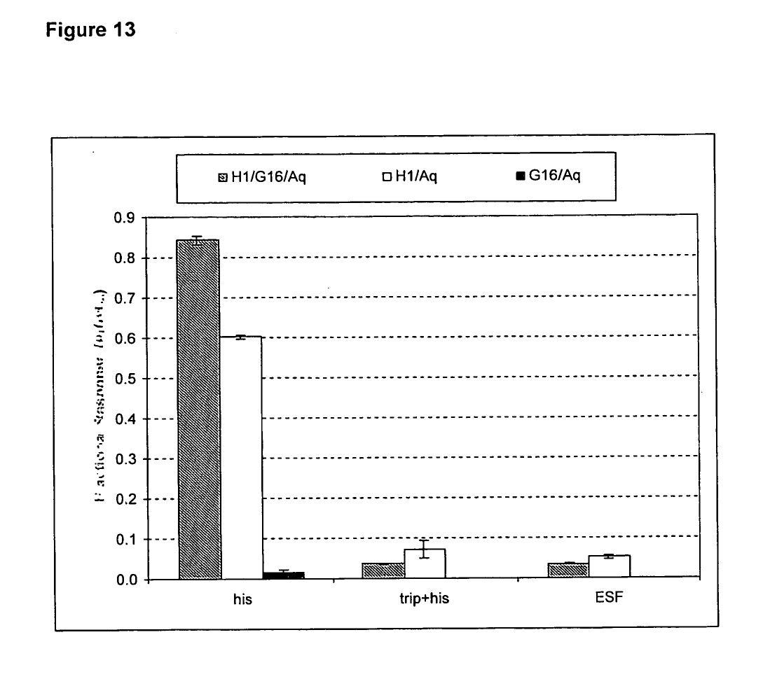

These experiments demonstrate that the human Histamine H1 GPCR is functionally expressed in insect cells. The net result of HRH1receptor activation in the insect assay cell line is activation of endogenous insect phospholipase Cbeta, which in turn leads to an increase in intracellular calcium. However, unlike the previous examples, Galpha₁₆ is not an absolute requirement for a functional response. Experiments with transiently transformed cell lines expressing various combinations of HH1R, Galpha₁₆ and aequorin are shown in FIG. 13. The results indicate that whilst the fractional response of assay cell lines to the endogenous agonist histamine is reduced in the absence of Galpha₁₆, it is still significant. By contrast, there is no response in the absence of the Histamine H1 receptor.

These results indicate that the human HH1 R receptor is able to couple to an endogenous hetero-trimeric G-protein in insect cells, one in which all three subunits are supplied by the insect. The G-alpha subunit in this endogenous insect G-protein is likely of the Gs-type, able to activate the effector enzyme adenyly cyclase and increase intracellular cAMP concentration, but NOT able to activate the effector enzyme phospholipase C beta and increase intracellular calcium concentration. Since the experiments above clearly show an increase in intracellular calcium concentration, as measured by activation of the calcium-sensitive photoprotein (apo)aequorin, then thismust have been caused by G-beta/gammasubunit activation of phospholipase C.

Thus we conclude that there is an endogenous G-protein G-beta-gammasubunit pair in insect cells which is capable of interacting with an endogenous G-alpha-s-subunit to form a functional heterotrimeric Gs-type G-protein. This insect Gs-type G protein can functionally interact with human GPCRs, and enables these receptors to induce an intracellular calcium flux on activation, as a result of G-beta-gammastimulation of phospholipase C, as well as a cAMP flux, as a result of G-alpha sstimulation of adenyly cyclase.

### EXAMPLE FOUR

### Human 5-HT2A GPCR

The cDNA for the human 5-Hydroxy tryptamine (5-HT2A) receptor (SwissProt 5H2A_HUMAN; AC P28223) was cloned into the insect expression vector p2Zop2F as described herein. The Galpha₁₆, and aequorin expression constructs were as described herein.

5H2A insect-based assay cell lines were created by simultaneous cotransfection of Sf9 insect cells with all three expression constructs as described herein. Transiently-transfected assay cell lines were used for the experiments described below. The human 5-HT2A GPCR is coupled in vivo to a Gq-type G-protein, which causes the activation of the effector enzyme phospholipase Cbeta, leading to an increase in the intracellular concentration of the second messenger calcium.

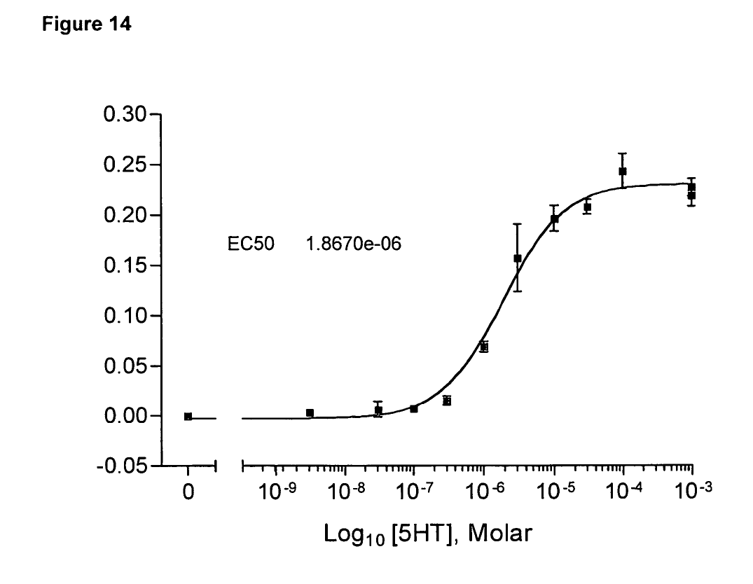

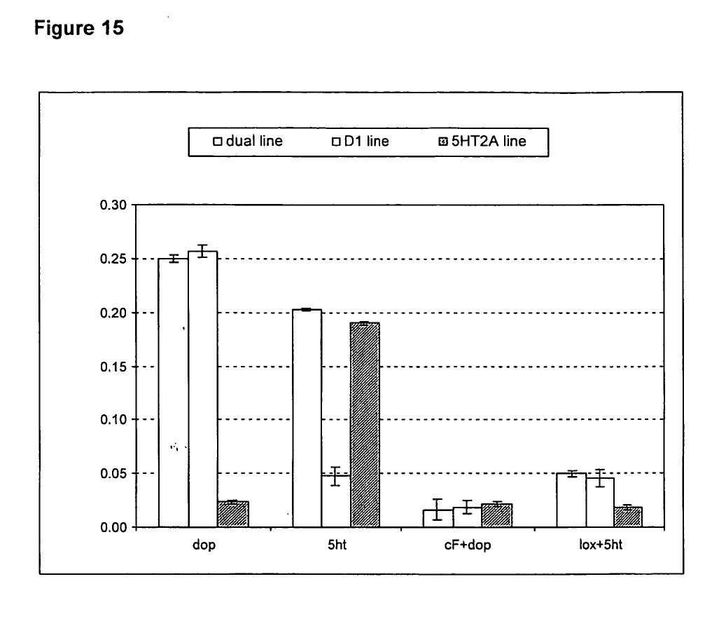

Transiently-transfected 5H2A assay cell lines showed a characteristic sigmoidal agonist concentration-activity curve when exposed to increasing concentrations of the endogenous agonist serotonin (FIG. 14), thus demonstrating functional coupling of the human 5H2A receptor to endogenous insect phospholipase Cbeta. Serotonin agonist activity can be blocked by pre-incubation of the 5H2C assay cell line with the antagonist loxapine (FIG. 15).

These experiments demonstrate that the human 5-HT2A GPCR is functionally expressed in insect cells, and able to signal through a hybrid G-protein consisting of human Galpha₁₆ and insect G beta gamma subunits. The net result of G protein activation in the insect assay cell line is activation of endogenous insect phospholipase Cbeta, which in turn leads to an increase in intracellular calcium.

Furthermore, it is possible to “multiplex” human GPCRs in the insect assay cell lines. FIG. 15 shows the results of a multiplex experiment in which the 5-HT2A and Dopamine D1A GPCRs were co-expressed in insect assay cell lines (together with Galpha₁₆ and aequorin; named the “dual line”). The responses of this multiplexed line to agonists and antagonists was compared with two other assay lines, each expressing only a single GPCR (5HT2A line or D1 line). The dual line shows a similar response to each of the single receptor lines in terms of agonist response; this indicates that in the multiplexed dual line, both receptors are functional and independent of each other.

### EXAMPLE FIVE

### Human 5-HT1A GPCR

The cDNA for the human 5-Hydroxytryptamine 1A (5-HT1A) receptor (SwissProt; 5H1A_HUMAN; AC P08908;) was cloned into the insect expression vector p2Zop2F as described herein. The Galpha₁₆, and aequorin expression constructs were as described herein.

5H1A insect-based assay cell lines were created by simultaneous cotransfection of Sf9 insect cells with all three expression constructs as described herein. Transiently-transfected assay cell lines were used for the experiments described below. The human 5-HT1A GPCR is coupled in vivo to a Gi-type G-protein, which causes the deactivation of the effector enzyme adenylyl cyclase, leading to an decrease in the intracellular concentration of the second messenger cAMP.

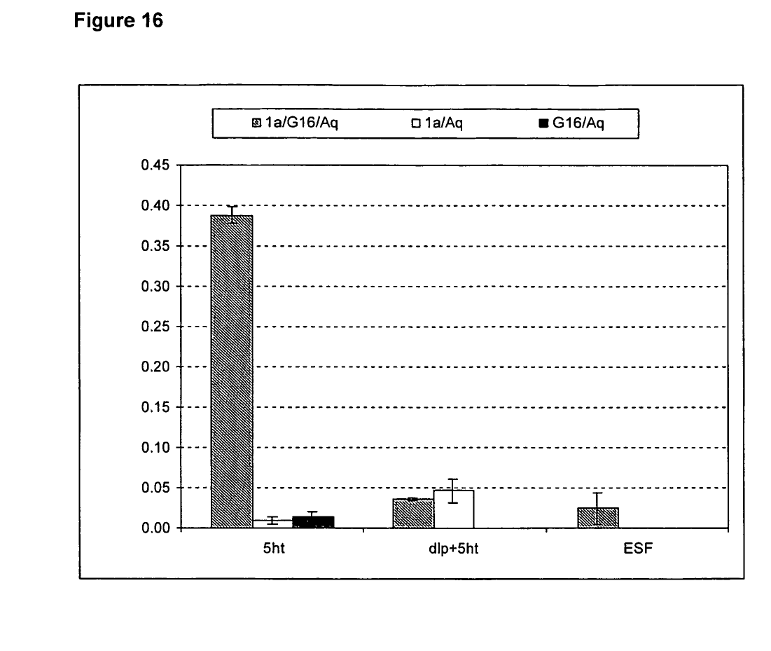

5H1A assay cells expressing all three assay components respond to the endogenous agonist, serotonin (5-HT; FIG. 16) at 100 microM concentration. Pre-incubation of the assay cells with the antagonist DL-propranalol (10 microM) prevents subsequent activation by serotonin. The Galpha₁₆, subunit is an absolute requirement for functional activity of the assay with the5H1A GPCR.

### EXAMPLE SIX

### Human Beta2 Adrenergic GPCR

The cDNA for the human beta2 Adrenergic receptor (SwissProt; B2AR_HUMAN; AC P07550; “B2AR”) was cloned into the insect expression vector p2Zop2F as described herein. The Galpah₁₆, and aequorin expression constructs were as described herein.

B2AR insect-based assay cell lines were created by simultaneous cotransfection of Sf9 insect cells with all three expression constructs as described herein. Transiently-transfected assay cell lines were used for the experiments described below. The human B2AR GPCR is coupled in vivo to a Gs-type G-protein, which causes the activation of the effector enzyme adenylyl cyclase, leading to an increase in the intracellular concentration of the second messenger cAMP.

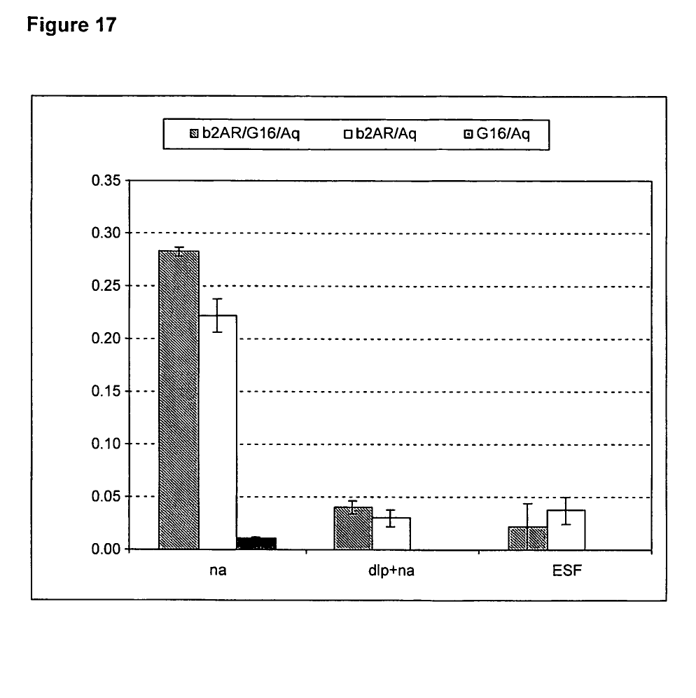

B2AR assay cells expressing all three assay components respond to the endogenous agonist, nor-epinephrine (FIG. 17) at 100 microM concentration. Pre-incubation of the assay cells with the antagonist DL-propranalol (10 microM) prevents subsequent activation by nor-epinephrine. The Galpha₁₆, subunit is not an absolute requirement for functional activity of the assay; although in its absence the response is reduced in magnitude.

### EXAMPLE SEVEN

### Human Muscarincic Acetylcholine M1 GPCR

The cDNA for the human Muscarincic Acetylcholine M1 receptor (SwissProt; ACM1_HUMAN; AC P11229;) was cloned into the insect expression vector p2Zop2F as described herein. The Galpha₁₆, and aequorin expression constructs were as described herein.

ACM1 insect-based assay cell lines were created by simultaneous cotransfection of Sf9 insect cells with all three expression constructs as described herein. Transiently-transfected assay cell lines were used for the experiments described below. The human ACM1 GPCR is coupled in vivo to a Gq-type G-protein, which causes the activation of the effector enzyme phospholipase Cbeta, leading to an increase in the intracellular concentration of the second messenger calcium.

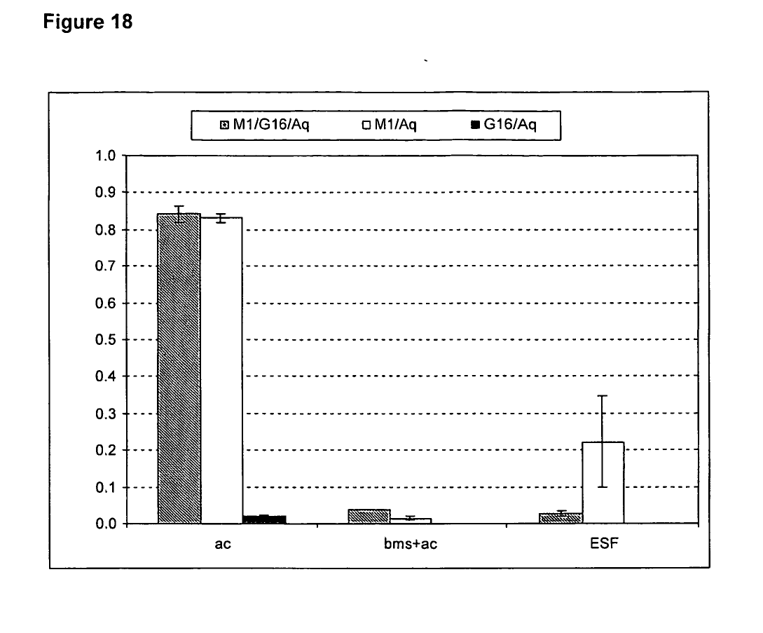

ACM1 assay cells expressing all three assay components respond to the endogenous agonist acetylcholine (FIG. 18) at 100 microM concentration. Pre-incubation of the assay cells with the antagonist 10 benztropine methanosulphate (10 microM) prevents subsequent activation by acetylcholine. The Galpha₁₆, subunit is not a requirement for functional activity of the assay; the acetylcholine agonist response is identical regardless of its presence or absence.

### REFERENCES

1. Wess, J. Molecular basis of receptor/G-protein-coupling selectivity. Pharmacology and Therapeutics 80, 231-264 (1998).

2. Dutton, G. Biotechnology companies increase their research on G-protein receptors. Genetic Engineering News 17, 8&34 (1997).

3. Wallace, R. W. & Goldman, M. E. Bioassay Design and Implementation. in High Throughput Screening (ed. Devlin, J. P.) 297-306 (Marcel Dekker, Inc., New York, 1997).

4. Miklos, G. L. G. & Rubin, G. M. The role of the genome project in determining gene function: insights from model organisms. Cell 86, 521-529 (1996).

5. Brody, T. & Cravchik, A. Drosophila melanogaster G protein-coupled receptors. The Journal of Cell Biology 150, F83-F88 (2000).

6. Quehenberger, O., Prossnitz, E. R., Cochrane, C. G. & Ye, R. D. Absence of Gi proteins in the Sf9 insect cell. Journal of Biological Chemistry 267, 19757-19760 (1992).

7. Amatruda III, T. T., Steele, D. A., Slepak, V. Z. & Simon, M. I. Ga16, a G protein a subunit specifically expressed in hematopoietic cells. Proceedings of the National Academy of Sciences USA 88, 5587-2291 (1991).

8. Prasher, D., McCann, R. O. & Cormier, M. J. Cloning and expression of the cDNA coding for Aequorin, a bioluminescent calcium-binding protein. Biochemical and Biophysical Research Communications 126, 1259-1268 (1985).

9. Offermanns, S. & Simon, M. I. Ga15 and Ga16 couple a wide variety of receptors to Phosopholipase C. Journal of Biological Chemistry 270, 15175-15180 (1995).

10. Jones, K., Hibbert, F. & Keenan, M. Glowing jellyfish, luminescence and a molecule called coelenterazine. Trends in Biotechnology 17, 477481 (1999).

11. Kendall, J. M. & Badminton, M. N. Aequorea victoria bioluminescence moves into an exciting new era. Trends in Biotechnology 16, 216-224 (1998).

12. Schmidt, B. Light Minded; Instruments for Chemiluminescence and Flourescence Measurements. The Scientist 13, 1823 (1999).

13. Farrel, L. M., Johnson, R. & latrou, K. A baculovirus (Bombyx mori nucleur polyhedrosis virus) repeat element functions as a powerful constitutive enhancer in trabsfected insect cells. Journal of Biological Chemistry 272, 30724-30728 (1997).

14. Graziano, M. P., Broderick, D. J. & Tota, M. R. Expression of G-Protein Coupled Receptors in Drosophila Schneider 2 Cells. in Identification and Expression of G Protein-Coupled Receptors (ed. Lynch, K. R.) Chapter 10, pp 181-195 (Wiley-Liss, Inc., 1998).

15. Pfeifer, T. A., Hegedus, D. D., Grigliatti, T. A. & Theilmann, D. A. Baculovirus immediate-early promoter-mediated expression of the Zeocin resistance gene for use as a dominant selectable marker in Dipteran and Lepidopteran insect cell lines. Gene 188, 183-190 (1997).

16. Hegedus, D. D., Pfeifer, T. A., Hendry, J., Theilmann, D. A. & Grigliatti, T. A. A series of broad host range shuttle vectors for constitutive and inducible expression of heterologous proteins in insect cell lines. Gene 207, 241-249 (1998).

17. Ungrin, M. D., Singh, L. M. R., Stocco, R., Sas, D. E. & Abramowitz, M. An automated aequorin luminescence-based functional calcium assay for G protein-coupled receptors. Analytical Biochemistry 272, 3442 (1998).

18. Banno, Y., Asano, T. & Nozawa, Y. Stimulation by G protein betagamma subunits of phospholipase C beta isoforms in human platelets. Thromb Haemost 79, 1008-1013 (1998).

19. Clapham, D. E. G protein bg subunits. Annual Review of Pharmacology and Toxicology 37, 167-203 (1997).

20. Harteneck, C., Plant, T. D. & Schultz, G. From worm to man: three subfamilies of TRP channels. Trends Neurosci 23, 159-166 (2000).

21. Rizzuto, R., Simpson, A. W. M., Brini, M. & Pozzan, T. Rapid changes of mitochondrial Ca2+ revealed by specifically targeted recombinant aequorin. Nature 358, 325-327 (1992).

22. Stables, J. et al. A bioluminescent assay for agonist activity at potentially any G-protein-coupled receptor. Analytical Biochemistry 252, 115-126 (1997).

23. Stadel, J. M., Wilson, S. & Bergsma, D. J. Orphan G protein-coupled receptors: a neglected opportunity for pioneer drug discovery. Trends in Pharmacological Sciences 18, 430437 (1997).

24. Pausch, M. H. G-protein-coupled receptors in Saccharomyces cerevisiae: high-throughput screening assays for drug discovery. Trends in Biotechnology 15, 487494 (1997).

25. Tate, C. G. & Grisshammer, R. Heterologous expression of G-protein-coupled receptors. Trends in Biotechnology 14, 426430 (1996).

What is claimed is:

1. A system for assaying G-protein-coupled receptor interactions, comprising:
a) an insect cell expressing a heterologous mammalian G-protein-coupled receptor expressed from a recombinant G-protein-coupled receptor coding sequence;
b) a heterologous G alpha protein expressed in the insect cell from a recombinant G alpha protein coding sequence;
c) one or more insect effector proteins that couple the heterologous G alpha protein to an endogenous insect signalling pathway, so that binding of a ligand to the heterologous G-protein coupled receptor mediates a detectable change in the insect cell.

2. The system of claim 1, wherein the recombinant G-protein-coupled receptor coding sequence is stably retained in the insect cell and subsequent generations of the insect cell.

3. The system of claim 1 or 2, wherein the recombinant G alpha protein coding sequence is stably retained in the insect cell and subsequent generations of the insect cell.

4. The system of any one of claims 1 through 3, wherein the detectable change in the insect cell is a calcium flux.

5. The system of any one of claims 1 through 4, wherein the insect cell expresses a plurality of different heterologous mammalian G-protein-coupled receptors, each of which is coupled to the endogenous insect signalling pathway.

6. The system of any one of claims 1 through 5, wherein the heterologous G-protein-coupled receptor is a human G-protein-coupled receptor.

7. The system of any one of claims 1 through 6, wherein the signalling pathway comprises an endogenous insect phospholipase.

8. The system of claim 7, wherein the endogenous insect phospholipase is a phospholipase-C beta.

9. The system of any one of claims 1 through 8, wherein the endogenous effector proteins comprise an endogenous insect G beta protein and an endogenous insect G gamma protein.

10. The system of any one of claims 1 through 9, wherein the insect cell is a lepidopteran cell.

11. The system of any one of claims 1 through 9, wherein the insect cell is a dipteran cell.

12. The system of any one of claims 1 through 9, wherein the insect cell is an Sf9 cell.

13. The system of any one of claims 1 through 9, wherein the insect cell is a Hi5 cell.

14. The system of any one of claims 1 through 13, wherein the heterologous G alpha protein is a G alpha 15 or G alpha 16 protein.

15. The system of claim 14, wherein the heterologous G-protein-coupled receptor couples in a natural state to a G protein other than G alpha 15 or G alpha 16.

16. The system of any one of claims 1 through 13, wherein the heterologous G alpha protein is selected from the group consisting of Gαolf, Gα12, Gα01, Gα15, Gαs, Gα13, Gαo2, Gα16, Gαi1, Gα11, Gαi2, Gαq, Gαi3, Gα14, Gαz.

17. The system of any one of claims 1 though 16, wherein the detectable change in the insect cell is a calcium flux, further comprising a heterologous calcium binding protein expressed in the insect cell from a recombinant calcium binding protein coding sequence, wherein the calcium binding protein is capable of providing a detectable signal that is indicative of the calcium flux in the insect cell.

18. The system of claim 17, wherein the calcium binding protein further comprises a mitochondrial peptide targeting signal.

19. The system of claim 17 or 18, wherein the calcium binding protein is Aequorin.

20. The system of any one of claims 1 through 19, wherein the detectable signal is a bioluminescent signal.

21. The system of any one of claims 1 through 20, further comprising a heterologous putative receptor ligand expressed in the insect cell from a putative receptor ligand coding sequence.

22. The system of any one of claims 1 through 21, further comprising a heterologous calcium ion channel expressed in the insect cell from a calcium ion channel coding sequence.

23. The system of claim 22, wherein the calcium ion channel is a TRP ion channel.

24. The system of claim 22, wherein the calcium ion channel coding sequence hybridizes under stringent conditions to the Drosophila ion channel trp coding sequence.

25. The system of any one of claims 1 through 16, wherein the detectable change in the insect cell is a calcium flux, wherein the calcium flux is detected by a fluorescent calcium sensitive molecule.

26. The system of claim 25, wherein the fluorescent calcium sensitive molecule is selected from the group consisting of Fluo 3 and Fluo 4.

27. An insect cell for assaying G-protein-coupled receptor interactions, comprising:
a) a heterologous mammalian G-protein-coupled receptor expressed in the insect cell from a recombinant G-protein-coupled receptor coding sequence;
b) a heterologous G alpha protein expressed in the insect cell from a recombinant G alpha protein coding sequence; and,
c) one or more endogenous effector proteins expressed in the insect cell that couple the heterologous G alpha protein to an endogenous signalling pathway, so that binding of a ligand to the heterologous G-protein coupled receptor mediates a detectable change in the insect cell.

28. A method for assaying G-protein-coupled receptor interactions, comprising:
a) expressing a heterologous mammalian G-protein-coupled receptor in an insect cell from a recombinant G-protein-coupled receptor coding sequence;
b) expressing a heterologous G alpha protein in the insect cell from a recombinant G alpha protein coding sequence;
c) expressing one or more endogenous effector proteins in the insect cell that couple the heterologous G alpha protein to an endogenous signalling pathway, so that binding of a ligand to the heterologous G-protein coupled receptor mediates a detectable change in the insect cell;
d) exposing the insect cell to the ligand; and,
e) detecting the detectable change in the insect cell.

29. A system for assaying G-protein-coupled receptor interactions substantially as hereinbefore described and with reference to the examples and drawings.


SEQUENCE LISTING (10 sequences)

SEQ ID NO: 1 - DNA, 23 bp, Artificial Sequence
  Description of Artificial Sequence Primer - D1F
atgaggactctgaacaactctgc

SEQ ID NO: 2 - DNA, 27 bp, Artificial Sequence
  Description of Artificial Sequence Primer - D1B
catggcagagttgttcagagtcctcat

SEQ ID NO: 3 - DNA, 33 bp, Artificial Sequence
  Description of Artificial Sequence Primer - ADBR2F
aagcttcaacatggggcaacccgggaacggcag

SEQ ID NO: 4 - DNA, 33 bp, Artificial Sequence
  Description of Artificial Sequence Primer-ADBR2R
tctagatttacagcagtgagtcatttgtactac

SEQ ID NO: 5 - DNA, 32 bp, Artificial Sequence
  Description of Artificial Sequence Primer - CHRM1F
aagcttaacatgaacacttcagccccacctgc

SEQ ID NO: 6 - DNA, 32 bp, Artificial Sequence
  Description of Artificial Sequence Primer - CHRM1R
uctagatttagcattggcgggagggagtgcgg

SEQ ID NO: 7 - DNA, 33 bp, Artificial Sequence
  Description of Artificial Sequence Primer - 5HT1AF
aagcttaacatggatgtgctcagcgctggtcag

SEQ ID NO: 8 - DNA, 32 bp, Artificial Sequence
  Description of Artificial Sequence Primer - 5HT1AR
tctagatttactggcggcagaacttacactta

SEQ ID NO: 9 - DNA, 32 bp, Artificial Sequence
  Description of Artificial Sequence Primer - HRH1F
aagcttacaatgagcctccccaattcctcctg

SEQ ID NO: 10 - DNA, 30 bp, Artificial Sequence
  Description of Artificial Sequence Primer - HRH1R
tctagatttaggagcgaatatgcagaattc